(19) United States
(12) Patent Application Publication
(10) Pub. No.: US 2026/0177552 A1
(43) Pub. Date: Jun. 25, 2026

(54) QUANTIFICATION OF IMMUNOLOGICAL STATUS AND TREATMENT PLANNING

(71) Applicant: Galvanize Therapeutics, Inc., Redwood City, CA (US)

(72) Inventors: Chiara PASTORI, Hayward, CA (US); Robert E. NEAL, II, Redwood City, CA (US)

(73) Assignee: Galvanize Therapeutics, Inc., Redwood City, CA (US)

(21) Appl. No.: 19/543,504

(22) Filed: Feb. 18, 2026

Related U.S. Application Data
(63) Continuation of application No. PCT/US2024/043295, filed on Aug. 21, 2024.
(60) Provisional application No. 63/578,134, filed on Aug. 22, 2023.
(60) Provisional application No. 63/597,639, filed on Nov. 9, 2023.

(51) Int. Cl. G01N 33/575; G01N 33/52

(52) U.S. Cl. CPC G01N 33/575; G01N 33/52; G01N 2333/90

(57) ABSTRACT

Methods, devices and systems are provided to analyze the immunologic response of patients at various times before, during and/or after immunomodulating therapy, particularly treatment with specialized pulsed electric field energy. In some embodiments, such methods, devices and systems include a laboratory developed test that utilizes a patient tumor biopsy sample and a series of blood draws to determine whether the locoregional therapy (with or without additional systemic therapies, or systemic therapies-alone) was sufficient to facilitate an increase in tumor-specific antibody production. In some embodiments, the focus is on evaluation of baseline antibody levels, prior to any treatment, and changes in levels of antibodies to given antigens, especially in response to a given therapy treatment. In such embodiments, this antibody testing is performed prior to treatment. The treatment, such as delivery of specialized PEF energy to a target tissue, is then performed and antibody levels are checked at one or more later timepoints to detect changes in the presence of antibodies and therefore response to therapy.

### CROSS REFERENCE TO RELATED APPLICATIONS

This application is a continuation of International PCT Patent Application No. PCT/US2024/043295, filed Aug. 21, 2024, which claims priority to and the benefit of U.S. Provisional Patent Application No. 63/597,639, filed Nov. 9, 2023, and U.S. Provisional Patent Application No. 63/578,134, filed Aug. 22, 2023, the entire contents of each of which are incorporated herein by reference.

### BACKGROUND

Abnormal tissue can take a variety of different forms, such as damaged, diseased, obstructive, cancerous or undesired tissue. In some instances, the abnormal tissue is a tumor, such as a benign tumor or a malignant tumor, a cyst, or an area of diseased tissue. One of the most troublesome types of abnormal tissue is related to cancer.

Cancer is a group of diseases characterized by the uncontrolled growth and spread of abnormal cells. If the spread is not controlled, it can result in death. Although the causes of cancer are not completely understood, numerous factors are known to increase the disease's occurrence, including many that are modifiable (e.g., tobacco use and excess body weight) and others that are not (e.g., inherited genetic mutations). These risk factors may act, simultaneously or in sequence, to initiate and/or promote cancer growth. Cancer is the second most common cause of death in the US, exceeded only by heart disease.

Lung, liver and pancreatic cancers are among the cancers having the lowest survival rates. Lung cancer is the leading cause of cancer death, more than colorectal, breast, and prostate combined. The overall change in 5-yr survival rate for all stages combined has only slightly improved over time: 1970's (approx. 13%), 2010's (approx. 17.2%), 2019 (approx. 21.7%). Liver cancer incidence rates have more than tripled since 1980, while the death rates have more than doubled during this time. Some progress has occurred in survival for patients with liver cancer, but 5-year survival remains low, even for those diagnosed at the localized stage. Pancreatic cancer is expected to be the 2nd leading cause of cancer-related death in 2020. The 5-yr survival rate for all stages is 9% and has not substantially improved over 40 years. These outcomes have endured despite the evolution of conventional therapies.

Many types of cancers are not successfully cured or recur at a later point in time. Recurrence typically occurs because the original treatment did not successfully eliminate all of the cancer cells and those left behind proliferated. In some instances, the cancer cells spread to other parts of the body in undetectable amounts, known as micrometastases. When these micrometastases are not overcome by the body, they grow to detectable levels and require additional treatment. And, ultimately, many patients lose their battle with cancer.

Consequently, improved therapies are needed that more successfully treat cancers and reduce or prevent their recurrence, along with improved therapies for all types of abnormal tissue. At least some of these objectives will be met by the present invention.

### SUMMARY OF THE INVENTION

Described herein are embodiments of apparatuses, systems and methods for treating target tissue in the body. Likewise, the invention relates to the following numbered clauses:

1. A method of determining an immunological status of a patient comprising:
obtaining a biospecimen having antibodies from the patient;
combining at least a portion of the biospecimen with at least one target antigen;
generating a colorimetric solution having an optical density that indicates an amount of the antibodies present in the at least a portion of the biospecimen that have bound to the at least one target antigen; and
determining the immunological status of the patient based at least in part on the amount.

2. A method as in clause 1, wherein generating a colorimetric solution comprises combining tagging antibodies with the amount of antibodies.

3. A method as in clause 2, wherein the tagging antibodies are specific to an antibody type IgA, IgM, IgG, or IgE.

4. A method as in clause 2, wherein generating the colorimetric solution comprises conjugating the tagging antibodies with an identifying material.

5. A method as in clause 4, wherein the identifying material comprises an enzyme.

6. A method as in clause 5, wherein the enzyme comprises horseradish peroxidase.

7. A method as in clause 4, wherein generating the colorimetric solution comprises combining a color revealing solution with the conjugated tagging antibodies so as to induce a colorimetric chemical reaction that colors or changes color of the color revealing solution.

8. A method as in clause 7, wherein a degree of optical density directly correlates to quantity of tagging antibody.

9. A method as in clause 7, further comprising measuring the optical density.

10. A method as in clause 9, further comprising measuring the optical density with a luminometer or spectrophotometer.

11. A method as in clause 1, wherein a higher optical density correlates to a higher amount of antibodies present.

12. A method as in clause 1, wherein the at least one target antigen is obtained from the patient.

13. A method as in clause 12, wherein the at least one target antigen is obtained from a tumor and the at least one target antigen is a tumor antigen.

14. A method as in clause 1, wherein the at least one target antigen is synthetically produced.

15. A method as in clause 1, wherein the biospecimen comprises blood.

16. A method as in clause 1, wherein determining the immunological status of the patient comprises determining a response of an immune system of the patient to a treatment.

17. A method as in clause 16, wherein the treatment comprises an immunomodulating therapy.

18. A method as in clause 17, wherein the immunomodulating therapy comprises delivery of pulsed electric field energy to a tissue comprising the at least one target antigen.

19. A method as in clause 18, wherein the tissue comprises a tumor and the at least one target antigen comprises a tumor antigen.

20. A method as in clause 17, wherein determining the response comprises determining if durable production of antibodies to the target antigen has been achieved.

21. A method as in clause 20, further comprising providing an additional treatment to the patient if durable production of antibodies to the target antigen has not been achieved.

22. A method as in clause 16, wherein determining the response comprises determining a type of antibody production.

23. A method as in clause 1, further comprising
obtaining another biospecimen having antibodies from the patient at a later time point than the step of obtaining the biospecimen having antibodies from the patient;
combining at least a portion of the another biospecimen with another at least one target antigen;
generating another colorimetric solution having an optical density that indicates another amount of the antibodies present in the at least a portion of the another biospecimen that have bound to the another at least one target antigen;
determining another immunological status of the patient based at least in part on the another amount; and
providing a treatment decision based on the determining of another immunological status.

24. A method of treating a patient comprising:
providing the treatment to the patient;
generating an evaluation of antibody production by the patient in response to the treatment; and
utilizing the evaluation to plan future treatment of the patient.

25. A method as in clause 24, wherein the evaluation comprises a quantification of the antibody production.

26. A method as in clause 24, wherein the evaluation comprises identification of one or more types of antibodies in the antibody production.

27. A method as in clause 24, wherein the treatment comprises delivery of pulsed electric field energy to the patient.

28. A method as in clause 27, wherein the pulsed electric field energy comprises packets of biphasic pulses.

29. A method as in clause 24, wherein generating an evaluation of antibody production comprises
obtaining a biospecimen from the patient after providing the treatment to the patient, wherein the biospecimen has antibodies;
combining at least a portion of the biospecimen with at least one target antigen; and
generating a colorimetric solution having an optical density that indicates an amount of antibodies present in the at least a portion of the biospecimen that have bound to the at least one target antigen thereby indicating antibody production.

30. A method as in clause 29, wherein generating a colorimetric solution comprises combining tagging antibodies with the amount of antibodies.

31. A method as in clause 30, wherein the tagging antibodies are specific to an antibody type IgA, IgM, IgG, or IgE.

32. A method as in clause 30, wherein generating the colorimetric solution comprises conjugating the tagging antibodies with an identifying material.

33. A method as in clause 32, wherein the identifying material comprises an enzyme.

34. A method as in clause 33, wherein the enzyme comprises horseradish peroxidase.

35. A method as in clause 32, wherein generating the colorimetric solution comprises combining a color revealing solution with the conjugated tagging antibodies so as to induce a colorimetric chemical reaction that colors or changes color of the color revealing solution.

36. A method as in clause 35, wherein a degree of optical density directly correlates to quantity of tagging antibody.

37. A method as in clause 35, further comprising measuring the optical density.

38. A method as in clause 37, further comprising measuring the optical density with a luminometer or spectrophotometer.

39. A method as in clause 29, wherein a higher optical density correlates to a higher amount of antibodies present.

40. A method as in clause 29, wherein the at least one target antigen is obtained from the patient.

41. A method as in clause 40, wherein the at least one target antigen is obtained from a tumor and the at least one target antigen is a tumor antigen.

42. A method as in clause 29, wherein the at least one target antigen is synthetically produced.

43. A method as in clause 29, wherein the biospecimen comprises blood.

44. A method as in clause 24, wherein the future treatment comprises additional delivery of pulsed electric field energy.

45. A method of quantifying an immunological response of a patient comprising:
obtaining a first biospecimen from the patient, wherein the first biospecimen comprises a target antigen;
obtaining a second biospecimen from the patient of the patient; and
combining at least a portion of the first biospecimen with at least a portion of the second biospecimen in a manner that produces a colorimetric solution that indicates a quantification of antibody if present that binds to the target antigen.

46. A method as in clause 45, wherein the colorimetric solution has an optical density that quantifies an amount of antibody present that has bound to the target antigen.

47. A method as in clause 45, further comprising delivering an immunomodulating therapy to the patient.

48. A method as in clause 47, wherein obtaining the second biospecimen occurs prior to delivering the immunomodulating therapy.

49. A method as in clause 48, further comprising obtaining a third biospecimen from the patient after delivering the immunomodulating therapy and combining a portion of the first biospecimen with a portion of the third biospecimen in a manner that produces another colorimetric solution that has another optical density that quantifies an amount of antibody in the third biospecimen that binds with the antigen of interest.

50. A method as in clause 49, further comprising comparing the optical density with the another optical density to determine an effectiveness of the immunomodulating therapy.

51. A method as in clause 47, wherein the immunomodulating therapy comprises delivery of pulsed electric field energy.

52. A method as in clause 51, wherein the pulsed electric field energy comprises packets of biphasic pulses.

53. A method of treating a patient comprising:
delivering pulsed electric field energy to a diseased tissue in the patient in a manner that causes an increase in antibody production by the patient;
obtaining a biospecimen from the patient, wherein the biospecimen comprises at least an antibody of the patient; and
quantifying an amount of antibody provided by the biospecimen.

54. A method for quantifying an antibody of interest comprising:
isolating a plurality of an antigen of interest;
obtaining a plurality of antibodies produced by a patient;
combining the plurality of antibodies with the plurality of the antigen of interest to allow the plurality of antibodies to bind with the plurality of the antigen of interest;
adding a plurality of tagging antibodies so that the plurality of tagging antibodies are allowed to bind with the plurality of antibodies; and
adding a solution that interacts with the plurality of tagging antibodies so as to induce a colorimetric chemical reaction which indicates the amount of bound tagging antibodies.

55. A system for determining an immunological status of a patient comprising:
at least one target antigen;
a plurality of tagging antibodies that are able to bind with antibodies of the patient that have bound with the at least one target antigen;
identifying material that is able to conjugate with the plurality of tagging antibodies; and
a color revealing solution combinable with the identifying material that has conjugated with the plurality of tagging antibodies so as to induce a colorimetric chemical reaction to create a colorimetric solution that has an optical density that indicates an amount of the antibodies that have bound to the at least one target antigen.

56. A system as in clause 55, wherein the at least one antigen is synthetically produced.

57. A system as in clause 55, wherein the tagging antibodies are specific to an antibody type IgA, IgM, IgG, or IgE.

58. A system as in clause 55, wherein the identifying material comprises an enzyme.

59. A system as in clause 58, wherein the enzyme comprises horseradish peroxidase.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C illustrate methods involving obtaining a biospecimen, lysing the cells of the biospecimen to obtain a solution of the proteins and plating the resulting solution in a well plate.

FIGS. 2A-2B schematically illustrate a single well of the well plate showing the isolation of bound antigens.

FIGS. 3A-3C illustrate methods involving obtaining a biospecimen from which antibodies are obtained, removing the cells from the biospecimen and plating the resulting solution in the well plate.

FIG. 4 schematically illustrates the single well of the well plate showing an antibody bound to an isolated bound antigen.

FIGS. 5A-5B illustrates a secondary antibody solution is added to the well plate.

FIG. 6 schematically illustrates a single well of the well plate showing the secondary antibody bound to the primary or patient antibody.

FIG. 7 schematically illustrates a single well of the well plate showing a solution added to the well that induces a colorimetric chemical reaction.

FIG. 8 illustrates a resultant solution of the colorimetric chemical reaction.

FIG. 9 provides a flowchart illustrating an embodiment of methodology for diagnosis and determining how likely a patient is to respond favorably to various therapies.

FIG. 10 provides a flowchart illustrating an embodiment of determining whether subsequent rounds of the same treatment would be beneficial, or whether additional or alternative forms of therapy should be explored if the effectiveness of a treatment is waning.

FIG. 11 provides a flowchart illustrating an embodiment of analyzing immunological status of more than one tumor in a patient.

FIG. 12 provides a flowchart illustrating an embodiment of determining immunostimulatory outcome.

FIGS. 13A-13B provide an overview illustration of an example therapeutic system for use in delivering specialized PEF energy.

FIGS. 14A-14C illustrate an example method of treatment.

FIGS. 15A-15B illustrate tissue lesions resulting from a study focusing on the effects of the addition of inter-cycle and inter-packet delays within the energy waveform on the local tissue characteristics.

FIG. 16 illustrates an embodiment of a waveform of a signal prescribed by an energy delivery algorithm.

FIG. 17 provides a table illustrating various example effects of parameter changes.

FIG. 18 illustrates antibody-dependent cell-mediated cytotoxicity pathways and complement-dependent cytotoxicity pathways.

FIGS. 19A-19C illustrate select results showing an increase in plasma cells, activated germinal B cells and memory B cells, respectively, in the mice that received the specialized PEF energy.

FIG. 20 illustrates a schematic of a custom ELISA workflow for serological analysis.

FIGS. 21A-21C illustrate results of a study indicating that treatment with specialized PEF stimulates the production of antibodies, similarly to a vaccination, that persist in blood for an extended period of time.

FIGS. 22A-22D illustrate results of a study indicating that the treatment with specialized PEF energy promotes more antibody production.

FIG. 23 summarizes IgG data from a 4T-1 tumor model and a EMT6 tumor models described herein, with the addition of IgG data from an EMT6 tumor model wherein blood was analyzed three months after treatment with specialized PEF energy.

### DETAILED DESCRIPTION OF THE INVENTION

Specific embodiments of the disclosed devices, systems, and methods will now be described with reference to the drawings. Nothing in this detailed description is intended to imply that any particular component, feature, or step is essential to the invention.

### I. Overview

The immune system utilizes antigens to determine which cells are part of the “self”′ and which are intruders. Antigens are usually proteins or polysaccharides found on the outside of cells and each has a unique shape that the immune system reads to identify whether the cell belongs in the body or not. Antigens exist on viruses, bacteria, allergens, parasites, proteins, tumor cells and normal cells in the body. Normal proteins in the body typically do not trigger an immune response because of self-tolerance, a process in which self-reacting cytotoxic T lymphocytes (CTLs) and autoantibody-producing B lymphocytes are culled “centrally” in primary lymphatic tissue (BM) and “peripherally” in secondary lymphatic tissue (mostly thymus for T-cells and spleen/lymph nodes for B cells). However, in some instances, proteins of the body do trigger an immune response. Examples of such proteins include any protein that is not typically exposed to the immune system, such as normal proteins that are well sequestered from the immune system, proteins that are normally produced in extremely small quantities, proteins that are normally produced only in certain stages of development, or proteins whose structure is modified due to mutation.

In tumors, genetic mutations in the cells accumulate, disrupting the downstream expressed proteins that ultimately manifest into uncontrolled cell growth that can spread throughout the body, such as in cancer. The mutated oncogenes thus produce proteins that behave differently than their natural form and typically trigger an immune response. These proteins (i.e. “antigens”) help the immune system fight against the cancer. Such antigens that are detected on cancer cells are known as “tumor-specific antigens” (TSAs), “tumor-associated antigens” (TAAs), neoantigens or oncogenic antigens, collectively referred to as tumor-antigens (TAs). The tumor antigens provide a cellular signature that identifies a cell as diseased. This signature can be detected by the immune system, informing them that they should kill these cells. Without recognition of these changes to the cellular phenotype and antigen expression, the immune system would not be able to recognize that the “self” cells are diseased.

The body is continually in a cancer immunoediting process which can be comprised of three phases: elimination, equilibrium, and escape. The elimination phase is when nascent tumor cells are successfully recognized and eliminated by the immune system, thus returning the tissues to their normal state of function. Tumor cells that elude the immunosurveillance phase will progress to the immune editing phase, called the equilibrium phase of advanced oncogenesis, where tumor expansion and metastasis are minimal (tumor dormancy) and usually occur without symptoms. In the equilibrium phase, the immune system may eventually eliminate all tumor cells leading to an outcome similar to the elimination phase. In a second scenario, the constant interaction of the immune system with tumors over a long period of time may actually “edit” or sculpt the phenotype of the developing tumor, resulting in the immunoselection of a tumor that has been shaped into a less-immunogenic state. Such tumors that are no longer susceptible to immune attack then progress into the immunoediting process, termed “escape.” The emergence of clinical symptoms of cancer generally correlates with the escape stage.

The status of the immunosurveillance process for a patient at any given time can be evaluated by analysis of the antibodies present in the patient. Antibodies are produced by B cells (specialized white blood cells). When an antigen comes into contact with a B cell, it causes the B cell to divide and clone. These cloned B cells- or plasma cells-release millions of antibodies into the bloodstream and lymph system and are specific to the antigen that stimulated their generation. When they find their paired antigen on the surface of a cell, they attach to it. An attached antibody serves as a red flag to the other cells of the immune system that the cell is diseased, marking it for destruction by the other cells of the immune system, including macrophage, natural killer cells and the complement system. After an infection or disease is cleared, the adaptive immune system has the benefit of retaining the educated cells to continue to patrol the body for signs of the disease again. These antibodies are able not only to clear the primary site of infection, but they can also monitor for recurrence of the disease, including target remote sites that a disease may have spread to. In cancer, this affords the invaluable ability to detect, target, and destroy metastases in advanced stage disease.

Antibodies, also known as immunoglobins, are categorized into five classes according to their location, constant region structure, and immune function. Each one is labeled by a letter, which is attached to an abbreviation of the term immunoglobulin (Ig): 1) IgA (found in saliva, tears, mucus, breast milk and intestinal fluid, IgA protects against ingested and inhaled pathogens), 2) IgD (found on the surface of B cells. Though its exact function is unclear, IgD is thought to support B cell maturation and activation, 3) IgE (found mainly in the skin, lungs and mucus membranes, IgE antibodies cause mast cells to release histamine and other chemicals into the bloodstream and are helpful for fighting off allergens 4) IgG (found mainly in blood and tissue fluids; this is the most common antibody, making up approximately 70% to 75% of all immunoglobulins in the body and protect the body from viral and bacterial infections, 5) IgM (found in blood and lymph system, IgM antibodies act as the first line of defense against infections and also play a large role in immune regulation).

Antibodies are proteins and each antibody has four polypeptides (peptides that consist of two or more amino acids), including two heavy chains and two light chains which join to form a Y-shaped molecule. The amino acid sequence in the tips of the “Y” varies greatly among different antibodies. This variable region, composed of 110-130 amino acids, gives the antibody its specificity for binding to the antigen that caused its generation. The variable region includes the ends of the light and heavy chains. The antibody also has a constant region that determines the mechanism used to bind or destroy the antigen when it comes in contact with the antigen. Thus, analysis of the antibodies of a patient at any given time can provide information on the types and amounts of tumor antigens present, which in turn provide information on the status of the ability of the patient to fight the cancer. Likewise, analysis of an antibody response by a patient at various time points could be utilized to monitor the mutational status of tumors within the patient.

Systems, methods, and devices are provided that enable rapid monitoring of a patient's immune response to a disease, such as cancer, and/or treatment of that disease. This data may be used as a diagnostic and prognostic indicator for how well the patient is inherently immunostimulated against the disease, their likelihood for responsiveness to an immunomodulating therapy, and evaluating their immune response to an immunomodulating therapy, to name a few. In some embodiments, an evaluation of the patient's immune response is made prior to treatment to identify the type(s) of cancer cells present and assess the patient's innate immune response. This can be used to determine the mutational status of the tumors or the individual mutational profiles of different tumors in the patient to understand their overlap or differences. Such knowledge can be used to guide the choice of treatment. Thus, such testing can be utilized for diagnosis and to determine how likely a patient is to respond favorably to various therapies. In some embodiments, an evaluation of the patient's immune response is made at one or more time points during a therapy protocol. This can be utilized to determine how well the patient is responding to the therapy. Likewise, such information can be utilized to determine if a change in therapy is desired, what such a change would comprise, and similar monitoring of the new therapy.

In some embodiments, the treatment itself has immunostimulatory effects. In such instances, the testing can be utilized to determine not only if the patient is responsive to the treatment and to what extent, but also the type of immunological response the treatment is eliciting. In some embodiments, the treatment includes specialized pulsed electric field (PEF) therapies as will be described in more detail herein. Such PEF therapies to generate immunogenic cell death in a targeted volume of tissue receiving the PEF energy by disrupting cellular homeostasis, resulting in a myriad of cell death processes and programmed cell death. Such cell death preserves the protein integrity, particularly stromal proteins comprising the extracellular matrix, because the mechanism of cell death does not rely on thermal processes. Furthermore, the tumor-specific and tumor-associated antigens (TAs) remain intact for antigen-presenting cells of the innate immune system to detect and educate the adaptive immune system. Thus, this treatment is unique and affords a superior safety profile in comparison to other therapies, such as surgical resection, radiation therapy, and thermal ablation.

The temporal and regional distribution of cell death processes provide a favorable cytokine tumor microenvironment for facilitating immune infiltration. This is achieved by release of intact immunogenic cell death markers and damage-associated molecular patterns (DAMPs), such as HMGB1, due to the cell death mechanism. The PEF energy also retains the supportive structure of the tissue (i.e. the collagen matrix) which permits access for immune infiltration. This is in contrast to cell death by thermal mechanisms which leave a coagulative necrotic environment preventing immunocyte access. These positive attributes of the specialized PEF energy and the steps of the cancer-immunity cycle have been extensively evaluated in preclinical studies, demonstrating an increase in treated and distant metastatic tumor response as a result of this virtuous cycle.

In some embodiments, it is desired to determine whether the adaptive immune cell responses invoked by the treatment include plasma cells producing antigen-specific IgG antibodies and cytotoxic (CD8+) T-cells that have been educated to antigens specific to the tumor. In a preclinical environment, both of these changes have been demonstrated using a tetramer specific to a known gp70 mutation in the murine cancer cell lines treated (4T1 and EMT6 orthotopic murine breast cancer).

Methods, devices and systems are provided to analyze the immunologic response of patients at various times before, during and/or after treatment, particularly treatment with specialized PEF energy. In some embodiments, such methods, devices and systems include a laboratory developed test that utilizes a patient tumor biopsy sample and a series of blood draws to determine whether the locoregional therapy (with or without additional systemic therapies, or systemic therapies-alone) was sufficient to facilitate an increase in tumor-specific antibody production.

In some embodiments, the focus is on evaluation of baseline antibody levels, prior to any treatment, and changes in levels of antibodies to given antigens, especially in response to a given therapy treatment. In such embodiments, this antibody testing is performed prior to treatment. The treatment, such as delivery of specialized PEF energy to a target tissue, is then performed and antibody levels are checked at one or more later timepoints to detect changes in the presence of antibodies. When performing this test, it is thus possible to preserve and “bank” primary biospecimens (i.e. the target tissue) and secondary biospecimens (i.e. blood samples). After collecting the desired number of biospecimens at the desired timepoints the antibody testing may be run in a single session if desired. Then, levels of pre-existing antibodies may be compared against the levels of antibodies after the intervention to discern if the patient is responding positively to the therapy. Alternatively, antibody levels may be evaluated in real time, and optionally compared to baseline levels. In such instances, the primary biospecimen is banked with preserving agents, fixation, refrigeration, or cryopreservation, and a portion of the primary biospecimen is utilized in the testing each time a new secondary biospecimen is collected and evaluated. Alternatively, the primary biospecimen may be processed to generate an antigen-containing plate wherein only some wells are used initially. The use of preserving agents, refrigeration, and other methods may be used to keep the remaining wells viable for testing with subsequent collections of the secondary biospecimen. In this way, real-time response levels may be collected, and long-term comparisons may be drawn over time. This approach is particularly valuable when discrete quantification of the antibody presence is available.

In some embodiments, the antibody testing methodologies are used to directionally advise a physician to the systemic adaptive immune response against the cancer that resulted from the therapy. This may be used to inform future care for the patient, such as delivering additional treatment sessions, integrating with other therapies, or transitioning to other therapy routes if they are unresponsive to the delivered therapy outcome. These decisions are able to be made faster than standard-of-care assessments that require imaging follow-up at 1 to 6 months following treatment therapy to monitor treated and distant tumor responses. By expediting the clinical decision-making process, care for patients would be improved and the cost of care delivery to the patient would be reduced by focusing their care on more effective approaches.

An additional use of the data gleaned may include indicators of specificity and robustness of the adaptive immune response for multi-metastatic disease and different mutational lineages of the cancer. For example, if a patient has five sites of tumor, and only two are able to be accessed in a given treatment session, if tumor-specific IgG antibody (TSA) presence becomes elevated in blood, and a 3rd tumor recedes, but two others do not, it could be presumed that the TAs associated with the treated tumors did not fully represent all tumor sites, and that an additional specialized PEF session at the residual tumor(s) would be desired to eliminate their patient burden. However, if treated tumors become stable or slightly regress, but residual tumors remain, and TSA presence is not elevated, then it may be assumed that the tumor responded to the ablative aspect of the therapy, but the immune upregulation was insufficient to produce detectable TSAs nor invoke a regression of any non-ablated tumors. This information could indicate additional ablations are required on the residual tumors or indicate the patient for different systemic therapies.

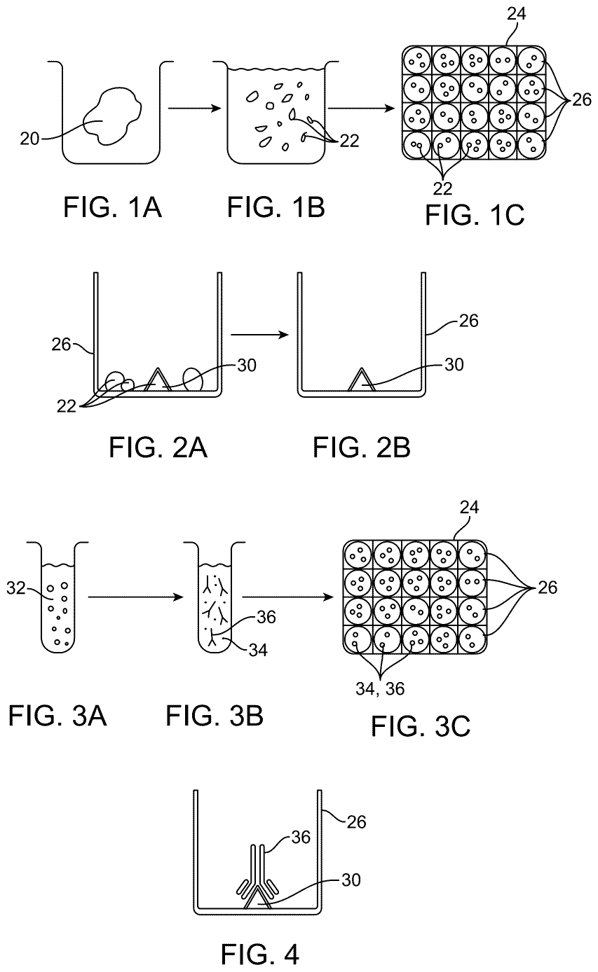

In some embodiments of the antibody test, a biospecimen 20 of the patient from the targeted region of interest, such as diseased tissue or a tumor, is collected via biopsy, resection, lavage, or blood draw, as appropriate. This is typically considered the primary biospecimen. This is illustrated in FIG. 1A. The biospecimen 20 is used to obtain the antigens, such as tumor antigens, involved in the patient's disease. This is achieved by methods described herein.

In one embodiment, the methods of obtaining antigens from the biospecimen 20 begins by lysing the cells of the biospecimen (FIG. 1B) to obtain a solution of the proteins 22, including the personalized tumor antigens. Typically, lysing is achieved by reagent-based methods. The reagents break the lipid barrier surrounding cells of the biospecimen by solubilizing proteins and disrupting lipid-lipid, protein-protein, and protein-lipid interactions. Through empirical testing, different detergent-based solutions composed of particular types and concentrations of detergents, buffers, salts, and reducing agents have been developed to provide the best possible results for particular species and types of cells. Detergents have both lysing and solubilizing effects.

The resulting solution is then placed within a well-plate 24 (FIG. 1C) that has been chemically treated to bind proteins, including the proteins 22 of the digested specimen solution. In some embodiments, all available protein types will adhere to the well plate 24. FIG. 2A schematically illustrates a single well 26 of the well plate 24 showing proteins 22 bound to the well 26. This may include tumor antigens 30 (represented by a triangular shape) and other types of proteins, such as collagen, elastin and other structural and cellular proteins. Since the tumor antigens 30 are of interest, at least some of the other proteins may optionally be washed away by additional steps (FIG. 2B) so as to diminish dilution of the tumor antigens 30. Any remaining solution is removed, and the wells are washed to remove any unbound material.

Referring to FIG. 3A, a secondary biospecimen 32 is then taken from the patient, this time the biospecimen 32 is utilized to obtain a sample of their antibodies. Thus, the secondary biospecimen 32 is typically from a blood draw (to obtain plasma or serum), additional tissue within or near tumors via biopsy or resection, or lavage. The secondary biospecimen 32 is processed depending on the type of specimen collected. If blood is collected and permitted to clot, the remaining liquid will be serum. If blood is collected in EDTA tubes, clotting will be prevented, and spinning the material leaves plasma. Both of these are viable for performing the test. If lavage or tissue samples are used, the material will be digested and centrifugated to remove the cellular constituents, leaving the interstitial protein compounds. In all secondary biospecimen types, the secondary biospecimen 32 is centrifugated and the residual fluid is collected for analysis, while the cells are discarded or used for other purposes. This fluid leaves the proteins 34 (including the antibodies 36) behind in a secondary biospecimen solution 37, as indicated in FIG. 3B.

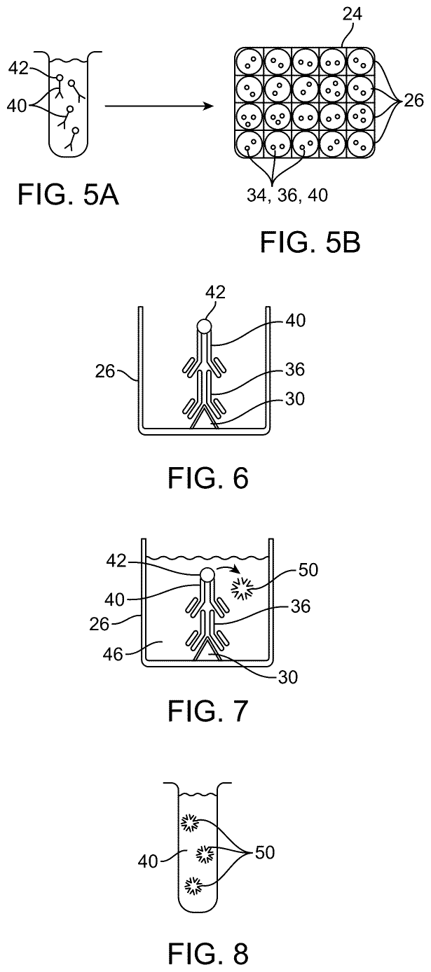

The secondary biospecimen solution 37 is then placed in the wells 26 containing the adhered antigens 30 and time is permitted for antibodies 36 in the solution to connect with any corresponding protein antigens 30 in the wells 26, as illustrated in FIG. 4. Because the vast majority of the patient proteins and cellular markers are recognized as “self”, there is typically no antibody 36 adherence to most of the plated antigens 30. However, if the plated antigens 30 contain the tumor antigen proteins, any antibodies 36 produced by the patient to target these tumor antigens 30 will adhere to the corresponding tumor antigens 30 in the wells 26 of the plate 24. Thus, any antibody 36 that adheres is targeting a known disease characteristic of the primary biospecimen 20. After sufficient time for initial antibody 36 connection, the secondary biospecimen solution 37 is removed. The next step of the method is undertaken to assist in identifying the antigens 30 present in the wells 26 for the purpose of quantification. In some embodiments, a tagging antibody 40 solution (FIG. 5A) is added to the well plate 24 (FIG. 5B). The tagging antibodies 40 are designed to bind to antibodies 36 and are conjugated with an identifying material 42, such as the enzyme horseradish peroxidase (HRP), for identification, as illustrated in FIG. 6. The tagging antibodies 40 are specific to the antibody type (e.g. IgA, IgM, IgG, IgE) and species of animal, but otherwise are non-specific and will bind to any available antibody 36 of that type and species. The bound tagging antibodies 40 thus reflect the presence of residual antibodies 36, which in turn reflect the presence of tumor antigens 30. After sufficient time for the tagging antibody 40 to bind to the antibody 36, the tagging antibody 40 solution is removed.

The next step of the method, as illustrated in FIG. 7, is undertaken to visualize the antibodies for quantification. A color revealing solution 46 is added to the wells 26. The color revealing solution 46 contains components to interact with the conjugated identifying material 42, inducing a colorimetric chemical reaction that changes the color 50 of the color revealing solution 46 (i.e. changes the color from clear or no color to a color having optical density). The solution 46 contains ample components to support the reaction. The degree of color 50 change is directly correlated with the amount of tagging antibody 40 present. The extent of color change of the color revealing solution 40 (FIG. 8) is then measured, such as by a luminometer/spectrophotometer which measures the optical density of the solution 40. This quantified metric indicates the relative amount of tagging antibody 40, which reflects the amount of antibodies 36 bound to the tumor antigens 30 in the well 26. This will be a result of both the total different tumor antigens 30 with affiliated antibodies 36 as well as the number of antibodies 36 in the systemic fluid targeting each of these tumor antigens 30. Thus, a greater color change value indicates a greater presence of anti-disease (anti-cancer) antibodies 36.

In some embodiments, absolute values of the tumor associated antigen presence are utilized rather than relative signal intensities. In some instances, absolute values are obtained from standard curves that are developed and incorporated into the assay. To develop a standard curve, a series of standard solutions with known concentrations of the target biomolecule are utilized. Typically, this involves a range of concentrations, often in a log scale, covering the expected range of concentrations in the biological specimen. For example, in some embodiments, standards with concentrations of 0, 1, 5, 10, 50, and 100 ng/mL of the target antibody are prepared and placed into separate wells for developing the signal intensity for each of these concentrations. The signal produced (e.g., absorbance or fluorescence) is measured in each well using a microplate reader as with the rest of this assay. The signal intensity is directly proportional to the amount of target biomolecule present in the well. Thus, when then analyzing the data from the test, plotting the known concentrations of the standard solutions (e.g x-axis) against their corresponding measured signals (e.g. y-axis) can be used to generate a curve using a linear regression model. Then, the signal from the experimental wells can be mapped to the standard curve, providing an absolute concentration of the target biomolecule in the sample. The standard curve may further be used to ensure the reliability of the ELISA results by checking for factors like assay linearity, precision, and accuracy.

It may be appreciated that in some instances the test protocol may be modified, such as to reduce or eliminate background noise in the antibody quantification. For example, there may be instances of contamination of the primary biospecimen 20, an infection nearby the tissue of the primary biospecimen 20, or other condition that may result in the presence of background non-self antigens present in the primary biospecimen that are not related to the cancer. In addition, there may be non-cancer antibodies present within the tumor environment in the primary biospecimen 20. Because the well plate is coated in a manner to allow binding of all proteins, and antibodies are inherently a protein, this could result in high background signal of antibodies. In some embodiments, to eliminate this background noise, the background noise is determined and then subtracted out from the well that contained the secondary biospecimen. In some embodiments, this is achieved by adding the tagging antibody 40 to the primary biospecimen 20 directly, without the secondary biospecimen 32 used. This tagging antibody 40 will thus bind to all available antibodies within the primary biospecimen 20 and is considered the background noise. This background noise is subtracted out from the well that contained the secondary biospecimen to provide targeted primary antibodies to the tumor antigens.

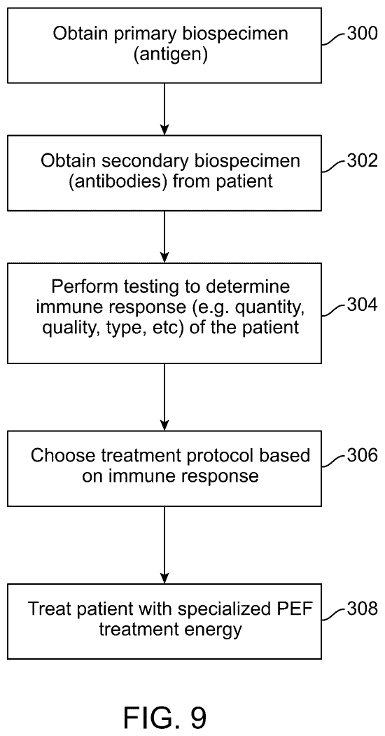

The above described methodologies provide a customized, patient-specific indicator of the patient's current immune response to their disease condition, particularly cancer. As mentioned, this may be utilized prior to treatment to establish an innate antibody response by the patient. Such knowledge can be used to guide the choice of treatment. Thus, such testing can be utilized for diagnosis and to determine how likely a patient is to respond favorably to various therapies. FIG. 9 provides a flowchart illustrating an embodiment of such methodology. Here, the method starts by obtaining the primary biospecimen (step 300). As described herein, the primary biospecimen is typically a sample of the tumor from the patient used to obtain the antigens, such as tumor antigens, involved in the patient's disease. It may be appreciated, however, that the primary biospecimen 20 may alternatively be obtained from other sources if a sample from the patient is not available. This is described in more detail herein and may include, for example, manufactured or synthetic versions. Next a secondary biospecimen 32 is obtained from the patient (step 302), such as blood containing antibodies. The secondary biospecimen 32 and a portion of the primary biospecimen 20 are then used to determine the immune response of the patient (step 304), such as according to the above-described methods (e.g. FIGS. 1A-1C, FIGS. 2A-2B, 3A-3C, FIG. 4, FIGS. 5A-5B, FIGS. 6-8). The information from the findings is then used to choose a treatment protocol based on the immune response (step 306). In this embodiment, the chosen treatment is with specialized PEF treatment energy (step 308).

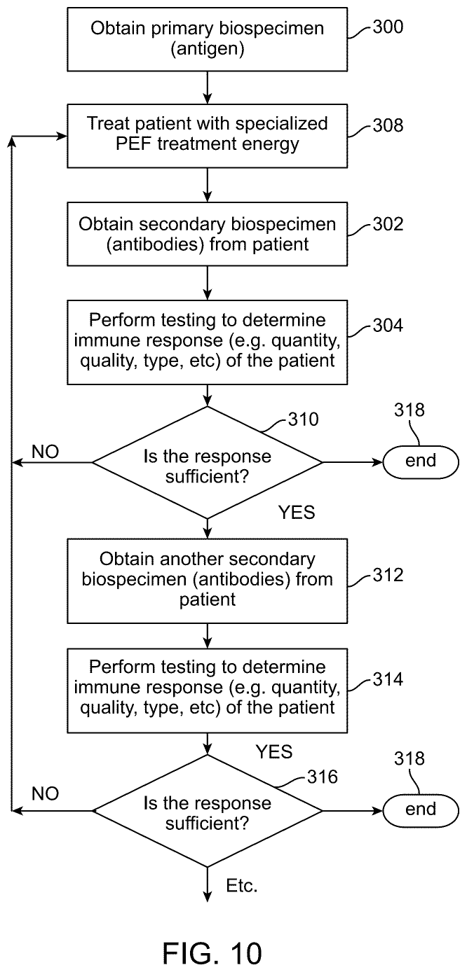

These (steps 300, 302, 304) or similar steps may be utilized at any point during treatment to determine the antibody production response the patient is having in response to the treatment. Thus, these methods (steps 300, 302, 304) can be used to track antibody levels over time for a patient. Because the tagging antibody 40 is type-specific (e.g. IgA, IgG, IgM, IgE), it also delineates which types of anti-cancer antibodies 36 are being produced. This information can be used to determine the quality (type) and quantity (amount) of antibody 36 production response they are having to the treatment(s). The antibody 36 production response is interpreted with knowledge of the biospecimen location and time point relative to treatment. The biospecimen location is relevant in that different antibodies may be present in different quantities depending on the biospecimen location, such as saliva versus blood. Likewise, different antibody types will have different response-curve profiles to a given treatment or disease presence, such as IgM being detectable prior to IgG antibodies, but IgG antibodies being longer-lived and thus more potent for long-term protection. The information obtained from the antibody testing methodologies may be particularly useful when the treatment includes therapies that facilitate antibody production. Examples of such therapies include specialized PEF therapies as described herein. Such monitoring can be used to ensure adequate antibody production is sustained over a targeted period, such as days, weeks, months, or years, in response to the therapy. This information can be used to determine whether subsequent rounds of the same treatment would be beneficial, or whether additional or alternative forms of therapy should be explored if the effectiveness of a treatment is waning. An example of such monitoring methodology is illustrated in FIG. 10. Here, the method starts by obtaining the primary biospecimen (step 300). As described herein, the primary biospecimen is typically a sample of the tumor from the patient used to obtain the antigens, such as tumor antigens, involved in the patient's disease. It may be appreciated, however, that the primary biospecimen 20 may alternatively be obtained from other sources if a sample from the patient is not available. This is described in more detail herein and may include, for example, manufactured or synthetic versions. Next the patient is treated (step 308), such as with the specialized PEF energy. Typically, this involves at least partially destroying the tumor. At a desired timepoint after the treatment, a secondary biospecimen 32 is obtained from the patient (step 302), such as blood containing antibodies. The secondary biospecimen 32 and a portion of the primary biospecimen 20 are then used to determine the immune response of the patient (step 304), such as according to the above-described methods (e.g. FIGS. 1A-1C, FIGS. 2A-2B, 3A-3C, FIG. 4, FIGS. 5A-5B, FIGS. 6-8). The information from the findings is then used to determine if the immune response was sufficient i.e. as desired (step 310). If the immune response is sufficient, additional time is allowed to pass. At another desired later timepoint, another secondary biospecimen 32′ is obtained from the patient (step 312), such as blood containing antibodies. The another secondary biospecimen 32′ and another portion of the primary biospecimen 20′ are then used to determine the immune response of the patient (step 314), such as according to the above-described methods (e.g. FIGS. 1A-1C, FIGS. 2A-2B, 3A-3C, FIG. 4, FIGS. 5A-5B, FIGS. 6-8). The information from the findings is then used to determine if the immune response was sufficient i.e. as desired (step 318). This can be repeated any desired number of times until the immune response is not found to be desired. At that point the PEF treatment can be performed again (step 308) and the flowchart is continued or the iterations end (step 318) wherein a different treatment may is performed or treatment is stopped.

Such methods may also be used to indicate the potential long-term success of the treatment where the adaptive immune response plays a crucial role in eliminating metastatic disease that cannot be detected (e.g. micrometastases) nor removed or destroyed in a way that fully ensures elimination of all residual patient cancer cells due to the sheer volume or number of metastases or safety hazards associated with eliminating the additional sites of cancer. In such instances, the long-term success of the treatment is reliant on the robustness of the immune system in relation to the disease.

In some embodiments, a variety of primary biospecimens are obtained from the patient, such as biospecimens from different tumors in the patient. The above-described laboratory methods (e.g. FIGS. 1A-1C, FIGS. 2A-2B, 3A-3C, FIG. 4, FIGS. 5A-5B, FIGS. 6-8) can then be undertaken to determine the patient's immune response status in relation to each tumor. Such information could be utilized to understand the differences between the tumors and any overlap. This information could then be used to determine the treatment plan, such as which areas to target and how. For example, the treatment plan may be based on which tumor region has the most broad-spectrum mutations wherein such a region would be the primary target. Or, for example, if a patient has five tumor masses and it is unclear which is the primary tumor versus metastases, or it is ambiguous to this aspect, any number of tumor masses may be sampled. It is possible that only three tumor masses are reachable for obtaining biospecimens. The biospecimens are then used to determine which of the tumors is responsive to the broadest antibody profile. This information can be used to derive decisions on which tumor to treat with an immunomodulating therapy, such as an antibody producing therapy. In some instances, it may be preferred to treat the tumor(s) with the most pronounced antibody response to obtain the biggest immune response spectrum. In other instances, it may be preferred to treat the tumor(s) with the least pronounced antibody response since treatment with an antibody producing therapy may increase the antibody production against the antigens in these specific individual masses. This information may also help guide a clinician as to whether all the masses should be treated or just a selected few of them.

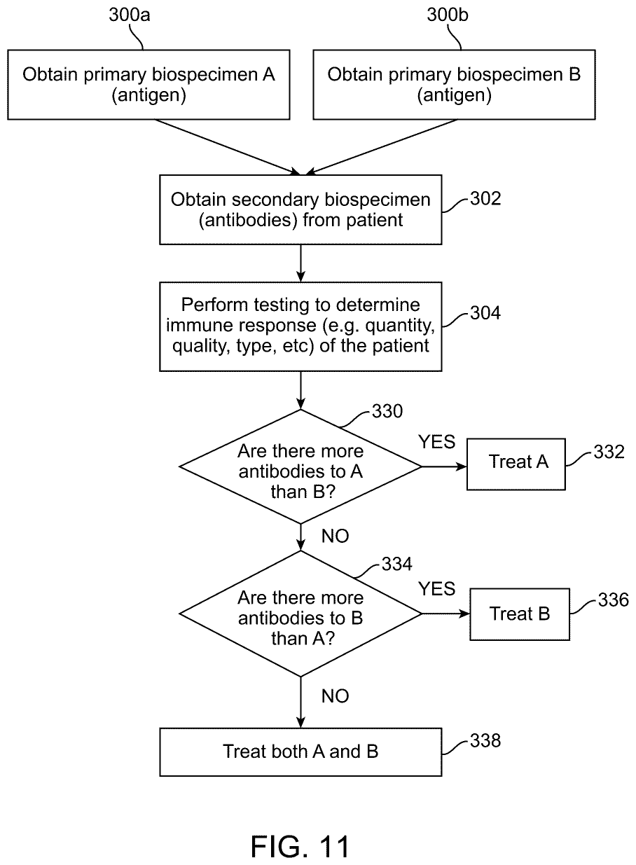

An example of such methodology is illustrated in FIG. 11. Here, the method starts by obtaining a primary biospecimen from a first tumor (step 300a) and a primary biospecimen from a second tumor (step 300b). As described herein, the primary biospecimen is typically a sample of the tumor from the patient used to obtain the antigens, such as tumor antigens, involved in the patient's disease. In this embodiment, the first tumor is the primary tumor and the second tumor is a metastasis. Next a secondary biospecimen 32 is obtained from the patient (step 302), such as blood containing antibodies. The secondary biospecimen 32 and a portion of the primary biospecimens from the first and second tumors are then used to determine the immune response of the patient (step 304) to each of the tumors, such as according to the above-described methods (e.g. FIGS. 1A-1C, FIGS. 2A-2B, 3A-3C, FIG. 4, FIGS. 5A-5B, FIGS. 6-8). The information from the findings is then used to compare the immune reaction to each of the tumors (step 332). In this embodiment, if there are more antibodies to the antigens of biospecimen A (the first or primary tumor) than to the antigens of biospecimen B (the second or metastatic tumor) then the first or primary tumor is treated (step 332). If not, further comparison of the immune reaction to each of the tumors (step 334) ensues. If there are more antibodies to the antigens of biospecimen B (the second or metastatic tumor) than to the antigens of biospecimen A (the first or primary tumor) then the second or metastatic tumor is treated (step 336). In this embodiment, if neither is the case, then both tumors are treated (step 338).

It may be appreciated that the above-described methods may also be used without the availability of an initial biospecimen from the patient, such as tumor tissue or diseased tissue. In some instances, manufactured versions of the mutated (e.g. cancerous) protein may be acquired (replacing the steps of FIGS. 1A-1B) and applied to the wells of the plate. This may be particularly the case when the mutations are common mutations in patient populations. However, if the mutations are not affiliated with commercially available proteins, specific proteins may be synthetically produced as needed that reflect the mutations. The methods then proceed as described in relation to FIGS. 2A-2B, FIGS. 3A-3C, FIG. 4, FIGS. 5A-5B, FIGS. 6-8. Thus, rapid monitoring of a patient's immune response to a disease, such as cancer, and/or treatment of that disease can still be undertaken if a biospecimen of the diseased tissue is unavailable.

In other embodiments, when a biospecimen of the diseased tissue is unavailable, a pre-assembled array of the most common or most probable antigens (for a given disease) are mixed and placed into the wells. These are commercially available for the most common ones, or a custom “cocktail” could be created for most probably mutations depending on a patient tumor type. For example, 20% of patients with non-small cell lung cancer (NSCLC) in the US will have an EGFR mutation, which would primarily entail one or more of four primary mutations (exon 19 deletion, T790M, L858R), which could all be sampled. NSCLC also often has KRAS, BRAF, and ALK rearrangements. Conversely, colorectal carcinoma shares some common mutations, such as KRAS and BRAF, but has many other common mutations not as frequently found in NSCLC, including APC (adenomatous polyposis coli), TP53, and PIK3CA genetic mutations. Thus, a panel of synthetically produced antigens for these mutations would be more valuable for colorectal cancer.

In addition to tumor-type, the custom-cocktails for common antigens may also be differentiated geographically or with race. For instance, while Caucasian NSCLC has 20% involvement with EGFR mutations, this same mutation is present in 50-60% of Asian and Pacific Islander populations, making it a more valuable one to include for this race than others. Further, patient demographics may also help ascribe specific antigen cocktails to test, such as ALK rearrangements being common in NSCLC non-smokers and light-smokers.

After acquiring the panel of synthetically produced common tumor antigens, the subsequent portions of the test proceed accordingly. as described in relation to FIGS. 3A-3C, FIG. 4, FIGS. 5A-5B, FIGS. 6-8. In this embodiment, even if the patient has the same mutated protein but the specific sequence of the mutation is different than the one acquired, there is a reasonable chance that the primary antibody will not bind to this different version of the antigen. In this instance, the antigens in the biomarker panel may differ from the actual antigens associated with the diseased tissue that the patient is carrying. This would potentially lead to false negatives, where the patient does have an increase in antibodies targeting a mutated gene, but because the specific mutation is different, it does not bind to the antigen in this test. Thus, due to the large array of potential oncogenes that may have antigenic mutations in different cancers, and the multiple varieties of mutations for each given gene, effective use of this approach would involve ensuring a sufficient variety of oncogenes and specific mutations of these oncogenes for the test to have sufficient analyte and clinical validity.

In another embodiment, when a biospecimen of the diseased tissue is unavailable, specific antigens or combinations of antigens are placed into the wells. The methods then proceed as described in relation to FIGS. 3A-3C, FIG. 4, FIGS. 5A-5B, FIGS. 6-8. If the patient has antibodies that bind to the antigens, it can then be discerned that the patient carries the antigens and antibodies against them. This information may be used to determine the likelihood of favorable or poor immunostimulatory responses for different treatments. This would afford a way to help guide downstream patient care. For example, if a treatment, such as PEF immunostimulation therapy were known to evoke strong anti-tumor antibody responses for patients with TP53 mutations, but not BRAF mutations, one could use the test described herein to delineate what the patient has developed pre-existing antibodies against, and then deduce that PEF therapy may be a good care option for the patient with the TP53 mutation, but not for the patient with BRAF mutation, as the invoked antibody response may be muted.

Likewise, using this test to discriminate patient outcomes and guide therapy may be linked to broader downstream outcomes than antibody level increases. For example, if it were known that EGFR mutations produce very strong anti-cancer antibody responses following an immunostimulatory therapy such as specialized PEF, but the KRAS mutations do not evoke this benefit, each mutated protein (or array of typical mutations for these proteins) would be placed into separate wells. One would then proceed with the methods of FIGS. 3A-3C, FIG. 4, FIGS. 5A-5B, and FIGS. 6-8 accordingly in each well. If antibodies were present for the EGFR-mutant well, one would know that the patient is likely to be a good responder to specialized PEF immunostimulation therapy. However, if the patient did not appear to have this mutation, but they had antibodies against the KRAS family of mutations, one would know they are less likely to respond and ascribe them to different care than specialized PEF. This could be done for other forms of immunomodulatory therapies as well, such as checkpoint inhibition.

In another embodiment, when it is known which antigens are most predictive of a positive therapeutic antibody response for a given therapy, such antigens can be placed into the wells. The methods then proceed as described in relation to FIGS. 3A-3C, FIG. 4, FIGS. 5A-5B, FIGS. 6-8. If the patient has antibodies that bind to the predictive antigens, this is an indication that the patient carries tumors with such antigens and the patient has a high likelihood of having a positive response to the proposed therapy. This may be used in determining the most effective treatment plan for the patient.

It may be appreciated that the above described methods may be utilized in conjunction with a variety of different treatment plans and therapies. Example therapies include but are not limited to tyrosine kinase inhibitors, specialized PEF energy, a combination of tyrosine kinase inhibitors/specialized PEF energy, chemotherapy, a combination of tyrosine kinase inhibitors/chemotherapy/specialized PEF, a combination of chemotherapy/specialized PEF, a combination of chemotherapy/immunotherapy, a combination of specialized PEF/immunotherapy, a combination of chemotherapy/specialized PEF energy/immunotherapy, etc.

In addition to alternate approaches for the primary specimen, it may be appreciated that other secondary biospecimens may be used to quantify the antibody presence. In some embodiments, such secondary biospecimens include whole blood, plasma, lavage contents, or liquid biopsies from areas of interest, such as the peripheral or inner regions of a tumor, lymph node, spleen, or other lymphatic structure, such as tertiary lymphoid structures.

It may be appreciated that the raw results from the antibody testing described herein may be analyzed via various approaches to determine targeted effect outcomes. For example, in addition to testing different antibody levels, the ratios of different antibodies (e.g. IgM v. IgG) may be used. Further, changes over different timepoints may be used to determine the relation of various outcomes, such as using changes in IgA or IgM antibodies between pre-treatment and 10±5 days post-treatment may be used as a very early indicator, while changes in IgG antibodies may be compared between pre-treatment and 30±10 days post-treatment for an indicator of more durable antibody production.

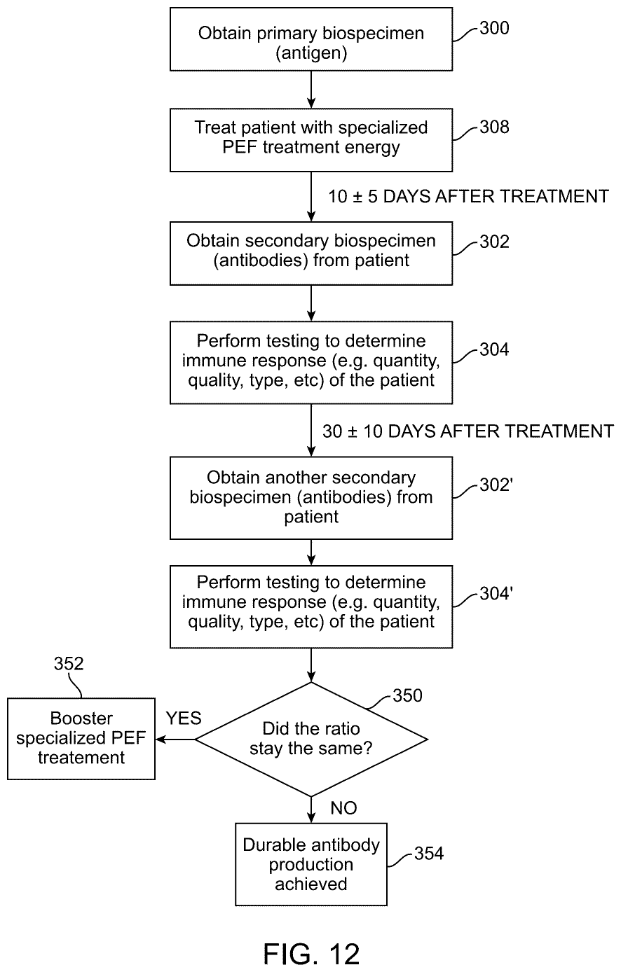

In addition, changes in IgM to IgG ratios between 10 and 30 days post-PEF may be used as a determinant of immunostimulatory outcome, giving a good indication for when additional specialized PEF deliveries are warranted. An example of such a method is illustrated in the flowchart of FIG. 12. Here, the method starts by obtaining the primary biospecimen (step 300). As described herein, the primary biospecimen is typically a sample of the tumor from the patient used to obtain the antigens, such as tumor antigens, involved in the patient's disease. It may be appreciated, however, that the primary biospecimen 20 may alternatively be obtained from other sources if a sample from the patient is not available. Next the patient is treated (step 308), such as with the specialized PEF energy. Typically, this involves at least partially destroying the tumor. At a desired timepoint after the treatment, a secondary biospecimen 32 is obtained from the patient (step 302), such as blood containing antibodies. In this embodiment, the desired timepoint is 10 days after the treatment, plus or minus 5 days. The secondary biospecimen 32 and a portion of the primary biospecimen 20 are then used to determine the immune response of the patient (step 304), such as according to the above-described methods (e.g. FIGS. 1A-1C, FIGS. 2A-2B, 3A-3C, FIG. 4, FIGS. 5A-5B, FIGS. 6-8).

At a later desired timepoint, another secondary biospecimen 32 is obtained from the patient (step 302′), such as blood containing antibodies. In this embodiment, the desired timepoint is 30 days after the treatment, plus or minus 10 days. The another secondary biospecimen 32 and a portion of the primary biospecimen 20 are then used to determine the immune response of the patient (step 304′), such as according to the above-described methods (e.g. FIGS. 1A-1C, FIGS. 2A-2B, 3A-3C, FIG. 4, FIGS. 5A-5B, FIGS. 6-8).

The information from the findings is then used to determine if the immune response was sufficient i.e. as desired (step 350), such as if durable antibody production was achieved.

For instance, if the IgM:IgG ratio is 10:1 at day −10 and persists at 10:1 at day −30, while absolute levels of IgM were decreasing, then it may be an indication that the IgM->IgG transition did not occur adequately, and additional “booster” doses of specialized PEF may be desired (step 352) to switch the immune response from short-lived to long-lived. Conversely, if IgM absolute levels decrease between day 10 and day 30, but the IgM:IgG ratio also changes from 10:1 at day −10 to 1:10 at day −30, then it may be presumed that the IgMs are lowering as a result of the IgM->IgG transition, and that a durable long-lived TA response is likely (step 354). This approach may similarly be used between other antibody population ratios and timepoints, which may afford even greater predictive value.

It may be appreciated that the antibody testing methodologies may be particularly useful when used in conjunction with immunomodulating therapies. As mentioned, one example of an immunomodulating therapy is treatment with specialized PEF energy. The specialized PEF energy and delivery has been optimized to provide advanced treatment of target tissue areas, including destruction of undesired tissue and generation of improved inflammatory and immune responses. These various types of treatment are controlled by a variety of factors including the electrode geometry, the dose of PEF energy delivered, the time the energy is delivered over, and the waveform of the PEF energy itself. The PEF energy is delivered in the form of a dose which is considered to be one application of the specialized energy. Each dose creates a lesion in the target tissue area. In some embodiments, the desired outcome is achieved with the delivery of a single dose. This achievement reduces treatment time and reduces any complications which may arise by providing additional doses, such as related to repositioning of the energy delivery device or retreatment of the same tissue area. The specific parameter values which define the waveform of the energy cause the waveform to be configured to deliver the desired effects with one dose. It may be appreciated that additional doses may be delivered to the target tissue if desired or the energy may be delivered to additional target tissues that may or may not include the original target tissue.

Devices, systems and methods described herein utilize a specific dose of specialized PEF energy to generate lesions having various zones, wherein manipulation of the zones provides benefits which culminate in a more comprehensive and therefore successful treatment of the patient, particularly when treating cancer and other diseases. The zones emanate from the delivery electrode radially outwardly, such as in rings. Each zone has differing cellular effects and therefore differing effects on the overall outcome of the treatment. In some instances, the zones include a cavitation zone, a thermal zone, a PEF zone, an inflammatory zone and an immune response zone. Typically, the zones are manipulated to eliminate or reduce the cavitation zone, eliminate or reduce the thermal zone and maximize the PEF zone along with a potential inflammatory zone and immune response zone. The size or amount of the zones are particularly generated so that their combination elicits an increase in the adaptive immune response of the patient, such as by promoting a cascade of increased recruitment for Tregs, CD4, CD8, and other adaptive immune cell types. These lesion classifications (i.e. zones) can be measured grossly, but also monitored in real-time based on the tissue properties (i.e. tissue density, impedance change, etc.) and perhaps most importantly, via imaging modalities such as CT, etc.

### II. Example Delivery Systems

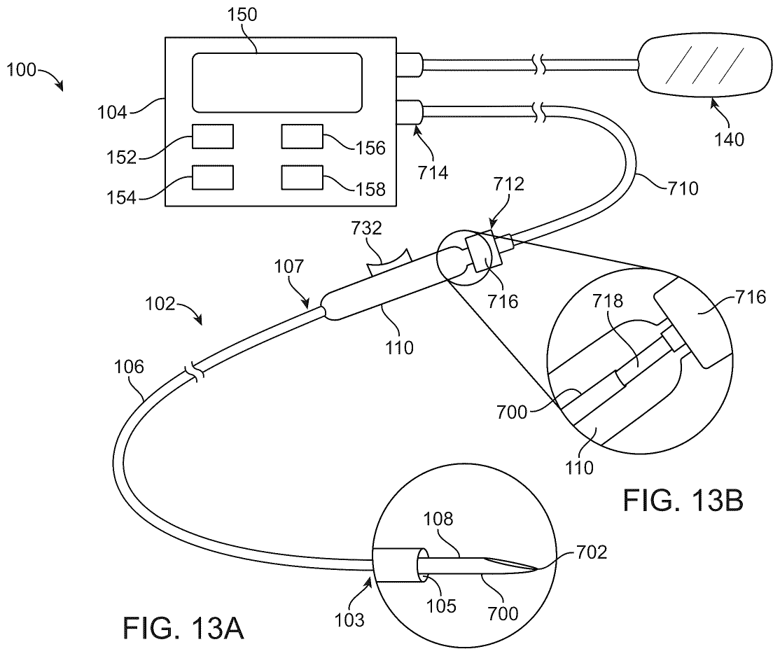

The specialized PEF energy is delivered with the use of systems and devices advantageously designed for superior access to target tissue throughout the body, particularly in locations previously considered inaccessible to percutaneous approaches. Such access is typically minimally invasive and relies on endoluminal approaches, though it may be appreciated that other approaches, such as percutaneous, laparoscopic or open surgical approaches, may be used in some situations, if desired. FIGS. 13A-13B provide an overview illustration of an example therapeutic system 100 for use in delivering the specialized PEF energy. In this embodiment, the system 100 comprises an elongate instrument 102 comprising a shaft 106 having a distal end 103 and a proximal end 107. The instrument 102 includes an energy delivery body 108 near the distal end 103 of the shaft 106. It may be appreciated that the energy delivery body 108 may take a variety of forms. The energy delivery body 108 may be mounted on or integral with an exterior of the shaft 106 so as to be externally visible. Or, the energy delivery body 108 may be housed internally within the shaft 106 and exposed by advancing from the shaft 106 or retracting the shaft 106 itself. Likewise, more than one energy delivery body 108 may be present and may be external, internal or both. In some embodiments, the shaft 106 is comprised of a polymer, such as an extruded polymer. It may be appreciated that in some embodiments, the shaft 106 is comprised of multiple layers of material with different durometers to control flexibility and/or stiffness. In some embodiments, the shaft 106 is reinforced with various elements such as individual wires or wire braiding. In either case, such wires may be flat wires or round wires. Wire braiding has a braid pattern and in some embodiments the braid pattern is tailored for desired flexibility and/or stiffness. In other embodiments, the wire braiding that reinforces the shaft 106 may be combined advantageously with multiple layers of material with different durometers to provide additional control of flexibility and/or stiffness along the length of the shaft.

In any case, each energy delivery body 108 comprises at least one electrode for delivery of the PEF energy. Typically, the energy delivery body 108 comprises a single delivery electrode and operates in a monopolar arrangement which is achieved by supplying energy between the energy delivery body 108 disposed near the distal end 103 of the instrument 102 and a return electrode 140 positioned upon the skin of the patient. It will be appreciated, however, that bipolar energy delivery and other arrangements may alternatively be used. When using bipolar energy delivery, the instrument 102 may include a plurality of energy delivery bodies 108 configured to function in a bipolar manner or may include a single energy delivery body 108 having multiple electrodes configured to function in a bipolar manner. The instrument 102 typically includes a handle 110 disposed near the proximal end 107. The handle 110 is used to maneuver the instrument 102, and typically includes an actuator 732 for manipulating the energy delivery body 108. In some embodiments, the energy delivery body 108 transitions from a closed or retracted position (during access) to an open or exposed position (for energy delivery) which is controlled by the actuator 732. Thus, the actuator 732 typically has the form of a knob, button, lever, slide or other mechanism. It may be appreciated that in some embodiments, the handle 110 includes a port for introduction of liquids, agents, substances, tools or other devices for delivery through the instrument 102. Example liquids include suspensions, mixtures, chemicals, fluids, chemotherapy agents, immunotherapy agents, micelles, liposomes, embolics, nanoparticles, drug-eluting particles, genes, plasmids, and proteins, to name a few.

The instrument 102 is in electrical communication with a generator 104 which is configured to generate the PEF energy. In this embodiment, the generator 104 includes a user interface 150, one or more energy delivery algorithms 152, a processor 154, a data storage/retrieval unit 156 (such as a memory and/or database), and an energy-storage sub-system 158 which generates and stores the energy to be delivered. In some embodiments, the user interface 150 on the generator 104 is used to select the desired treatment algorithm 152. In other embodiments, the algorithm 152 is automatically selected by the generator 104 based upon information obtained by one or more sensors. A variety of energy delivery algorithms may be used. In some embodiments, one or more capacitors are used for energy storage/delivery, however any other suitable energy storage element may be used. In addition, one or more communication ports are typically included.

The distal end 103 of the instrument 102 is typically advanceable through a delivery device, such as an endoscope. Endoscopes typically comprise a control body attached to an elongate insertion tube having a distal tip. The endoscope has an interior lumen accessible by a port into which the distal end 103 of the instrument 102 passes. The shaft 106 of the instrument 102 advanceable through the interior lumen and exits out of the distal tip of the endoscope. Imaging is achieved through the endoscope with the use of a light guide tube having an endoscopic connector which connects to a light and energy source. The distal tip of the endoscope may be outfitted with visualization technologies including but not limited to video, ultrasound, laser scanning, etc. These visualization technologies collect signals consistent with their design and transmit the signal either through the length of the shaft over wires or wirelessly to a video processing unit. The video processing unit then processes the video signals and displays the output on a screen. It may be appreciated that the endoscope is typically specific to the anatomical location to which it is being used, such as gastroscopes (upper GI endoscopy, which includes the stomach, esophagus, and small intestine (duodenum)), colonoscopes (large intestine), bronchoscopes (lungs), laryngoscopes (larynx), cystoscopes (urinary tract), duodenoscopes (small intestine), enteroscopes (digestive system), ureteroscopes (ureter), hysteroscopes (cervix, uterus), etc. It may be appreciated that in other embodiments, the instrument 102 is deliverable through a catheter, sheath, introducer, needle or other delivery system.

Endoluminal access allows treatment of target tissue from within various lumens in the body. Lumens are the spaces inside of tubular-shaped or hollow structures within the body and include passageways, canals, ducts and cavities to name a few. Example luminal structures include blood vessels, esophagus, stomach, small and large intestines, colon, bladder, urethra, urinary collecting ducts, uterus, vagina, fallopian tubes, ureters, kidneys, renal tubules, spinal canal, spinal cord, and others throughout the body, as well as structures within and including such organs as the lung, heart and kidneys, to name a few. In some embodiments, the target tissue is accessed via the nearby luminal structure. In some instances, a treatment instrument 102 is advanced through various luminal structures or branches of a luminal system to reach the target tissue location. For example, when accessing a target tissue site via a blood vessel, the treatment instrument 102 may be inserted remotely and advanced through various branches of the vasculature to reach the target site. Likewise, if the luminal structure originates in a natural orifice, such as the nose, mouth, urethra or rectum, entry may occur through the natural orifice and the treatment instrument 102 is then advanced through the branches of the luminal system to reach the target tissue location. Alternatively, a luminal structure may be entered near the target tissue via cut-down or other methods. This may be the case when accessing luminal structures that are not part of a large system or that are difficult to access otherwise.

Once a target tissue area has been approached endoluminally, energy can be delivered to the target tissue in a variety of ways. In one arrangement, an energy delivery body 108 is positioned within a body lumen and energy is delivered to the target tissue that is has entered the body lumen, through at least a portion of the lumen wall to target tissue either within the lumen wall and/or at least partially surrounding the lumen wall or through the lumen wall to target tissue outside and nearby the lumen wall. In another arrangement, the energy delivery body 108 is advanced through the lumen wall and inserted within or near target tissue outside of the lumen wall. It may be appreciated that such arrangements may be combined, involving at least two energy delivery bodies 108, one positioned within the body lumen and one extending through the wall of the body lumen. In some embodiments, each of the energy delivery bodies 108 function in a monopolar manner (e.g. utilizing a return electrode placed at a distance). In other embodiments, at least some of the energy delivery bodies 108 function in a bipolar manner. Since the lumen itself is preserved throughout the treatment, these delivery options are possible and allow treatment of tissue in, on or nearby the lumen itself. Such delivery of therapy allows access to previously inaccessible tissue, such as tumors or diseased tissue that has invaded lumen walls or has wrapped at least partially around a body lumen, too close to be surgically removed or treated with conventional focal therapies. Many conventional focal therapies, such as treatment with thermal energy, damage or destroy the structure of the lumen walls due to thermal protein coagulation, etc. In particular, bowel injuries caused by radiofrequency ablation are one of the most feared complications and have been associated with mortality due to sepsis and abscess formation. Consequently, most physicians will defer radiofrequency ablation in tumors adjacent to bowel. Other conventional focal therapies are ineffective near particular body lumens. For example, cryotherapy relies on sufficient cooling of tissue which is compromised by flow through body lumens, such a blood through the vasculature, which reduces the cooling effects. Such endoluminal access is also less invasive than other types of treatment, such as percutaneous delivery of energy involving the placement of numerous needle probes through the skin and deeply into tissues and organs. Since natural openings in the body are utilized, less wound healing is incurred along with reduced possible points of infection. Likewise, locations deep within the body can be access along with locations that are difficult to otherwise access from the outside, such as locations behind other organs or near great vessels, etc. It may be appreciated that a variety of anatomical locations may be treated with the systems and methods described herein. Examples include luminal structures themselves, soft tissues throughout the body located near luminal structures and solid organs accessible from luminal structures, including but not limited to liver, pancreas, gall bladder, kidney, prostate, ovary, lymph nodes and lymphatic drainage ducts, underlying musculature, bony tissue, brain, eyes, thyroid, etc. It may also be appreciated that a variety of tissue locations can be accessed percutaneously.

The endoscopic approach also lends itself to monopolar energy delivery. As mentioned, monopolar delivery involves the passage of current from the energy delivery body 108 (near the distal end of the instrument 102) to the target tissue and through the patient to a return pad 140 positioned against the skin of the patient to complete the electric current circuit. Thus, in some embodiments, the instrument 102 includes only one energy delivery body 108 or electrode. This allows the instrument 102 to have a low profile so as to be positionable within smaller body lumens. This also allows deep penetration of tissue surrounding the energy delivery body 108. Likewise, when penetrating the lumen wall with such devices, only one penetration is needed per treatment due to the use of only one energy delivery body 108. It may be appreciated that additional penetrations may occur due to various device designs or treatment protocols, however in some embodiments, the monopolar delivery design reduces the invasiveness of the procedure, simplifies the device and treatment design and provides superior treatment zones in target tissue.

The devices, systems and methods described herein may be used on their own or in combination with other treatments. Such combinatory treatment may be applicable to cancer treatment in particular. For example, the PEF treatment described herein may be used in combination with a variety of non-surgical therapies, neoadjuvant and adjuvant therapies such as radiotherapy, chemotherapy, targeted therapy/immunotherapy, focal therapy, gene therapy, plasmid therapy, to name a few. Example focal therapies include microwave ablation, radiofrequency ablation, cryoablation, high intensity focused ultrasound (HIFU), and other pulsed electric field ablation therapies. Such combination may condition the tissue for improved responsiveness and in some cases a synergistic response that is greater than either of the therapies alone. In addition, the PEF treatments described herein may lead to an abscopal effect due to the nature of the therapy.

FIGS. 13A-13B, illustrate a therapeutic system 100 comprising an energy delivery catheter 102 connectable with a generator 104. As shown, the catheter 102 comprises a shaft 106 having a distal end 103, a proximal end 107 and at least one lumen 105 extending at least partially therethrough. Likewise, the catheter 102 also includes at least one energy delivery body 108. In this embodiment, an energy delivery body 108 has the form of a probe 700 that is disposed within the lumen 105 of the shaft 106. The probe 700 has a probe tip 702 that is advanceable through the lumen 105 and extendable from the distal end 103 of the shaft 106 (expanded in FIG. 13A to show detail). In this embodiment, the tip 702 has a pointed shape configured to penetrate tissue, such as to resemble a needle. Thus, in this embodiment, the probe tip 702 is utilized to penetrate the lumen wall W and surrounding tissue so that it may be inserted into the target tissue external to the body lumen. Thus, the probe 700 has sufficient flexibility to be endoluminally delivered yet has sufficient column strength to penetrate the lumen wall W and target tissue. In some embodiments, the catheter 102 has markings to indicate to the user the distance that the probe tip 702 has been advanced so as to ensure desired placement.

In some embodiments, the probe extends from the distal end 103 of the shaft 106 approximately less than 0.5 cm, 0.5 cm, 1 cm, 2 cm, 3 cm, 4 cm, 5 cm, 6 cm, 7 cm, 8 cm or more than 8 cm. In some embodiments, the probe extends 1-3 cm or 2-3 cm from the distal end of the shaft 106. In some embodiments, the probe is 18 gauge, 19 gauge, 20 gauge, 21 gauge, 22 gauge, 23 gauge, 24 gauge, or 25 gauge. In some embodiments, the probe 700 is comprised of a conductive material so as to serve as an electrode. Thus, the electrode would have the size of the exposed probe. Example materials include stainless steel, nitinol, cobalt-chromium alloy, copper, and gold. In some embodiments, the exposed probe conductive material is coated with a different material, with examples including platinum-iridium, gold, platinum black, palladium, or other materials. The conductive material, or the conductive material coating may be designed so as to reduce the biological interactions of the tissue, reduce the production of electrochemical effects from the PEF treatment, or more efficiently distribute the PEF energy into the tissue, among other purposes. The materials may be smooth, electropolished, sandblasted at various grits, or treated with other mechanical or chemical preparations to alter the roughness of the surface, which may be done to reduce biological interactions of the tissue, facilitate easier deployment and retraction of the electrode, reduce the production of electrochemical effects from the PEF treatment, or more efficiently distribute the PEF energy into the tissue, among other purposes. Thus, in these embodiments, the PEF energy is transmittable through the probe 700 to the probe tip 702. Consequently, the shaft 106 is comprised of an insulating material or is covered by an insulating sheath. Example insulating materials include polyimide, silicone, polytetrafluoroethylene, and polyether block amide. The insulating material may be consistent or varied along the length of the shaft 106 or sheath. Likewise, in either case, the insulating material typically comprises complete electrical insulation. However, in some embodiments, the insulating material allows for some leakage current to penetrate.

When the probe 700 is energized, the insulting shaft 106 protects the surrounding tissue from the treatment energy and directs the energy to the probe tip 702 (and any exposed portion of the probe 700) which is able to deliver treatment energy to surrounding tissue. Thus, the tip 702 acts as a delivery electrode and its size can be selected based on the amount of exposed probe 700. Larger electrodes can be formed by exposing a greater amount of the probe 700 and smaller electrodes can be formed by exposing less. In some embodiments, the exposed tip 702 (measured from its distal end to the distal edge of the insulating shaft) during energy delivery has a length of 0.1 cm, 0.2 cm, 0.3 cm, 0.4 cm, 0.5 cm, 0.6 cm, 0.7 cm, 0.8 cm, 0.9 cm, 1 cm, 2 cm, 3 cm, greater than 3 cm, up to 8 cm, less than or equal to 0.1 cm, less than or equal to 0.3 cm, less than or equal to 0.5 cm, less than or equal to 1 cm, 0.2-0.3 cm, 0.1-0.5 cm, 0.1-1 cm, and all ranges and subranges therebetween. In addition to changing the size of the electrode, the tip 702 is retractable into the shaft 106 to allow for atraumatic endoscopic delivery and is then advanceable as desired to reach the target tissue. In this embodiment, advancement and retraction are controlled by an actuator 732 (e.g. knob, button, lever, slide or other mechanism) on a handle 110 attached to the proximal end 107 of the shaft 106. It may be appreciated that the shaft 106 itself may be advanced toward the target tissue, with or without advancing the probe from the distal end 103 of the shaft 106. In some embodiments, the distal end of the shaft 106 is advanced up to 20 cm into the tissue, such as from an external surface of a luminal structure or from an external surface of the body of the patient.

The handle 110 is connected to the generator 104 with the use of a specialized energy plug 510. The energy plug 510 has a first end 512 that connects to the handle 110 and a second end 514 the connects to the generator 104. The connection of the first end 512 with the handle 110 is expanded for detail in FIG. 13B. In this embodiment, the first end 712 has an adapter 716 that includes a connection wire 718 extending therefrom. The connection wire 718 is insertable into the proximal end of the probe 700 within the handle 110. This allows the energy to be transferred from the generator 104, through the connection wire 718 to the probe 700. Thus, the probe 700 is able to be electrified throughout its length, however only the exposed tip 702 delivers energy to the tissue due to the presence of the insulated shaft 106.

In this embodiment, the generator 104 includes a user interface 150, one or more energy delivery algorithms 152, a processor 154, a data storage/retrieval unit 156 (such as a memory and/or database), and an energy-storage sub-system 158 which generates and stores the energy to be delivered. In some embodiments, one or more capacitors are used for energy storage/delivery, however any other suitable energy storage element may be used. In addition, one or more communication ports are included.

In some embodiments, the generator 104 includes three sub-systems: 1) a high-energy storage system, 2) a high-voltage, medium-frequency switching amplifier, and 3) the system controller, firmware, and user interface. The system controller includes a cardiac synchronization trigger monitor that allows for synchronizing the pulsed energy output to the patient's cardiac rhythm. The generator takes in alternating current (AC) mains to power multiple direct current (DC) power supplies. The generator's controller can cause the DC power supplies to charge a high-energy capacitor storage bank before energy delivery is initiated. At the initiation of therapeutic energy delivery, the generator's controller, high-energy storage banks and a bi-phasic pulse amplifier can operate simultaneously to create a high-voltage, medium frequency output.

It will be appreciated that a multitude of generator electrical architectures may be employed to execute the energy delivery algorithms. In particular, in some embodiments, advanced switching systems are used which are capable of directing the pulsed electric field circuit to the energy delivering electrodes separately from the same energy storage and high voltage delivery system. Further, generators employed in advanced energy delivery algorithms employing rapidly varying pulse parameters (e.g., voltage, frequency, etc.) or multiple energy delivery electrodes may utilize modular energy storage and/or high voltage systems, facilitating highly customizable waveform and geographical pulse delivery paradigms. It should further be appreciated that the electrical architecture described herein above is for example only, and systems delivering pulsed electric fields may or may not include additional switching amplifier components.

The user interface 150 can include a touch screen and/or more traditional buttons to allow for the operator to enter patient data, select a treatment algorithm (e.g., energy delivery algorithm 152), initiate energy delivery, view records stored on the storage/retrieval unit 156, and/or otherwise communicate with the generator 104. The user interface 150 can include a voice-activated mechanism to enter patient data or may be able to communicate with additional equipment in the suite so that control of the generator 104 is through a secondary separate user interface.

In some embodiments, the user interface 150 is configured to receive operator-defined inputs. The operator-defined inputs can include a duration of energy delivery, one or more other timing aspects of the energy delivery pulse, power, and/or mode of operation, or a combination thereof. Example modes of operation can include (but are not limited to): system initiation and self-test, operator input, algorithm selection, pre-treatment system status and feedback, energy delivery, post energy delivery display or feedback, treatment data review and/or download, software update, or any combination or subcombination thereof.

In some embodiments, the system 100 also includes a mechanism for acquiring an electrocardiogram (ECG), such as an external cardiac monitor 170. Example cardiac monitors are available from AccuSync Medical Research Corporation. In some embodiments, the external cardiac monitor 170 is operatively connected to the generator 104. The cardiac monitor 170 can be used to continuously acquire an ECG signal. External electrodes 172 may be applied to the patient P to acquire the ECG. The generator 104 analyzes one or more cardiac cycles and identifies the beginning of a time period during which it is safe to apply energy to the patient P, thus providing the ability to synchronize energy delivery with the cardiac cycle. In some embodiments, this time period is within milliseconds of the R wave (of the ECG QRS complex) to avoid induction of an arrhythmia, which could occur if the energy pulse is delivered on a T wave. It will be appreciated that such cardiac synchronization is typically utilized when using monopolar energy delivery, however it may be utilized as part of other energy delivery methods.

In some embodiments, the processor 154, among other activities, modifies and/or switches between the energy-delivery algorithms, monitors the energy delivery and any sensor data, and reacts to monitored data via a feedback loop. In some embodiments, the processor 154 is configured to execute one or more algorithms for running a feedback control loop based on one or more measured system parameters (e.g., current), one or more measured tissue parameters (e.g., impedance), and/or a combination thereof.

The data storage/retrieval unit 156 stores data, such as related to the treatments delivered, and can optionally be downloaded by connecting a device (e.g., a laptop or thumb drive) to a communication port. In some embodiments, the device has local software used to direct the download of information, such as, for example, instructions stored on the data storage/retrieval unit 156 and executable by the processor 154. In some embodiments, the user interface 150 allows for the operator to select to download data to a device and/or system such as, but not limited to, a computer device, a tablet, a mobile device, a server, a workstation, a cloud computing apparatus/system, and/or the like. The communication ports, which can permit wired and/or wireless connectivity, can allow for data download, as just described but also for data upload such as uploading a custom algorithm or providing a software update.

The data storage/retrieval unit 156 can be, for example, a random access memory (RAM), a memory buffer, a hard drive, a database, an erasable programmable read-only memory (EPROM), an electrically erasable read-only memory (EEPROM), a read-only memory (ROM), flash memory, and/or so forth. The data storage/retrieval unit 156 can store instructions to cause the processor 154 to execute modules, processes and/or functions associated with the system 100.

Some embodiments the data storage/retrieval unit 156 comprises a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) can be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as ASICs, Programmable Logic Devices (PLDs), Read-Only Memory (ROM) and Random-Access Memory (RAM) devices. Other embodiments described herein relate to a computer program product, which can include, for example, the instructions and/or computer code discussed herein.

Examples of computer code include, but are not limited to, micro-code or micro-instructions, machine instructions, such as produced by a compiler, code used to produce a web service, and files containing higher-level instructions that are executed by a computer using an interpreter. For example, embodiments can be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

In some embodiments, the system 100 can be communicably coupled to a network, which can be any type of network such as, for example, a local area network (LAN), a wide area network (WAN), a virtual network, a telecommunications network, a data network, and/or the Internet, implemented as a wired network and/or a wireless network. In some embodiments, any or all communications can be secured using any suitable type and/or method of secure communication (e.g., secure sockets layer (SSL)) and/or encryption. In other embodiments, any or all communications can be unsecured.

As described herein, a variety of energy delivery algorithms 152 are programmable, or can be pre-programmed, into the generator 104, such as stored in memory or data storage/retrieval unit 156. Alternatively, energy delivery algorithms can be added into the data storage/retrieval unit to be executed by processor 154. The processor 154 can be, for example, a general-purpose processor, a field programmable gate array (FPGA), an application specific integrated circuit (ASIC), a digital signal processor (DSP), and/or the like. The processor 154 can be configured to run and/or execute application processes and/or other modules, processes and/or functions associated with the system 100, and/or a network associated with the system 100. As used herein the term “module” refers to any assembly and/or set of operatively-coupled electrical components that can include, for example, a memory, a processor, electrical traces, optical connectors, software (executing in hardware), and/or the like. For example, a module executed in the processor can be any combination of hardware-based module (e.g., a FPGA, an ASIC, a DSP) and/or software-based module (e.g., a module of computer code stored in memory and/or executed at the processor) capable of performing one or more specific functions associated with that module.

Each of these algorithms 152 may be executed by the processor 154. In some embodiments, the instrument 102 includes one or more sensors 160 that can be used to determine temperature, impedance, resistance, capacitance, conductivity, pH, optical properties (coherence, echogenicity, fluorescence), electrical or light permittivity, and/or conductance, to name a few. In some embodiments, one or more of the electrodes act as the one or more sensors. In other embodiments, the one or more sensors are separate from the electrodes. It may be appreciated that one or more sensors 160 may be disposed in a variety of locations, particularly depending on the parameter being sensed. For example, a sensor may be located along an energy delivery body 108, along an interior of the instrument, along the shaft 106, along an element that protrudes from the instrument 120, etc. Multiple sensors 160 may be present for sensing the same parameter at multiple sites, sensing different parameters at different sites, or sampling parameters at different sites to compile a single metric value measurement (e.g. average temperature, average voltage exposure, average conductivity, etc). One or more sensors 160 may alternatively or additionally be located on a separate device. Sensor data can be used to plan the therapy, monitor the therapy and/or provide direct feedback via the processor 154, which can then alter the energy-delivery algorithm 152. For example, impedance measurements can be used to determine not only the initial dose to be applied but can also be used to determine the need for further treatment, or not.

It will be appreciated that the system 100 can include an automated treatment delivery algorithm that could dynamically respond and adjust and/or terminate treatment in response to inputs such as temperature, impedance at various voltages or AC frequencies, treatment duration or other timing aspects of the energy delivery pulse, treatment power and/or system status.

In some embodiments, imaging is achieved with the use of a commercially available system, such as an endoscope connected with a separate imaging screen. It will be appreciated that imaging modalities can be incorporated into the instrument 102 or used alongside or in conjunction with the instrument 102. The imaging modality can be mechanically, operatively, and/or communicatively coupled to the instrument 102 using any suitable mechanism.

### III. Intra-Luminal Placement and Energy Delivery

As mentioned previously, in one arrangement, an energy delivery body 108 is positioned within a body lumen and energy is delivered to or through the lumen wall to target tissue either within the lumen, within the lumen wall, at least partially surrounding the lumen wall or outside the lumen wall. Thus, the target tissue is able to be treated from an energy delivery body 108 positioned within a body lumen.

In some embodiments, the treatment devices and systems are configured for luminal access and delivery of therapeutic energy toward the luminal walls so as to treat the nearby target tissue. The therapeutic energy is generally characterized by high voltage pulses which allow for removal of target tissue with little or no destruction of critical anatomy, such as tissue-level architectural proteins among extracellular matrices. This prevents dangerous collateral effects, such as stenosis, thrombus formation or fistulization, to name a few, and also allows for regeneration of healthy new luminal tissue within days of the procedure. Examples of systems which provide this type of therapeutic treatment include the pulmonary tissue modification systems (e.g., energy delivery catheter systems) described in commonly assigned patent applications including international patent application number PCT/US2017/039527 titled “GENERATOR AND A CATHETER WITH AN ELECTRODE AND A METHOD FOR TREATING A LUNG PASSAGEWAY,” which claims priority to U.S. provisional application Nos. 62/355,164 and 62/489,753, international patent application number PCT/US2018/067501 titled “METHODS, APPARATUSES, AND SYSTEMS FOR THE TREATMENT OF DISORDERS” which claims priority to U.S. Provisional Application No. 62/610,430, and international patent application number PCT/US2018/067504 titled “OPTIMIZATION OF ENERGY DELIVERY FOR VARIOUS APPLICATIONS” which claims priority to Provisional Patent Application No. 62/610,430 filed Dec. 26, 2017 and U.S. Provisional Patent Application No. 62/693,622 filed Jul. 3, 2018, all of which are incorporated herein by reference for all purposes.

As mentioned previously, one or more energy delivery algorithms 152 are programmable, or can be pre-programmed, into the generator 104 for delivery to the patient. The one or more energy delivery algorithms 152 specify electric signals which provide energy delivered to the lumen walls which are non-thermal (e.g. below a threshold for thermal ablation; below a threshold for inducing coagulative thermal damage), reducing or avoiding inflammation, and/or preventing denaturation of stromal proteins in the luminal structures. In general, the algorithm 152 is tailored to affect tissue to a pre-determined depth and/or to target specific types of cellular responses to the energy delivered. It may be appreciated that depth and/or targeting may be affected by parameters of the energy signal prescribed by the one or more energy delivery algorithms 152, the design of the instrument 102 (particularly the one or more energy delivery bodies 108), and/or the choice of monopolar or bipolar energy delivery. Typically, depths of up to 0.01 cm, up to 0.02 cm, 0.01-0.02 cm, up to 0.03 cm, 0.03-0.05 cm, up to 0.05 cm, up to 0.08 cm, up to 0.09 cm, up to 0.1 cm, up to 0.2 cm, up to 0.5 cm, up to 0.7 cm, up to 1.0 cm, up to 1.5 cm, up to 2.0 cm, up to 2.5 cm, up to 3.0 cm, up to 3.5 cm, up to 4.0 cm, up to 4.5 cm, or up to 5.0 cm, to name a few. These depths may be larger for circumferentially focal targets, or they may exist for entire circumferential depths through the lumen and parenchymal tissue.

Thus, the treatment is minimally invasive, quickly and easily executable, and has relatively low sensitivity to electrode placement (e.g. due to the monopolar arrangement) therefore allowing technicians of various skill levels to achieve high levels of consistency as well as successful outcomes. In some embodiments, the monopolar arrangement is possible without the need for muscular paralytics due to the waveform characteristics of the energy used. This can mitigate muscle contractions from motor neuron and skeletal muscle depolarization to an acceptable level, with or without a neuromuscular paralytic. Thus, it becomes possible to implement monopolar-directed treatment delivery through a lumen out to a distant pad, producing a more predictable and desirable treatment zone. It may be appreciated that paralytics may optionally be used depending on the type of energy and the depth of penetration desired.

### IV. Extra-Luminal Placement and Energy Delivery

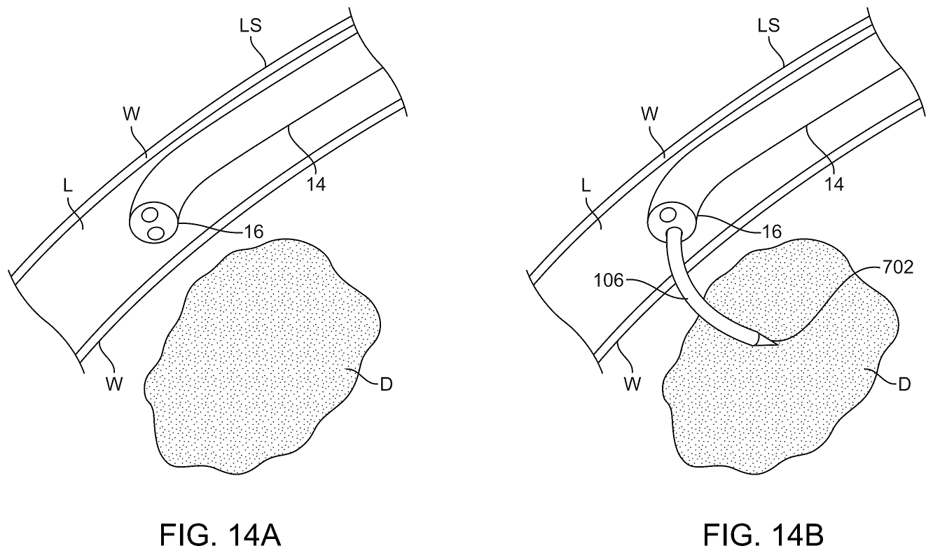

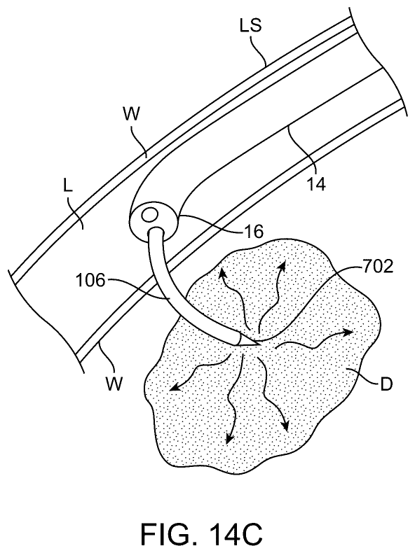

FIGS. 14A-14C illustrate an example method of treatment. FIG. 14A illustrates abnormal or diseased tissue D, such as a tumor, near a luminal structure LS. In this example, the diseased tissue D is near the luminal structure LS but spaced a distance from the lumen wall W. This luminal structure LS is used to access and the diseased tissue D and extra-luminally treat the diseased tissue D near the luminal structure LS. In this embodiment, the elongate insertion tube 14 of an endoscope 10 is advanced into the luminal structure LS and its distal tip 16 is steered toward the lumen wall W, beyond which lies the diseased tissue D. Once desirably positioned, the treatment catheter 102 is advanced through a lumen in the insertion tube 14 so that the distal end 103 of the shaft 106 extends beyond the tip 16 of the endoscope 10, as illustrated in FIG. 14B. In this embodiment, the probe tip 702 assists in penetrating the wall W and the shaft 106 is advanced across the wall W until the probe tip 702 is desirably positioned within the diseased tissue D. Referring to FIG. 14C, in this embodiment, the probe tip 702 is then advanced from the shaft 106 so as to create a desired delivery electrode size. Energy is then delivered according to one or more energy delivery algorithms 152, through the probe 700 to the diseased tissue D, as illustrated in FIG. 14C by wavy arrows extending radially outwardly from the probe tip 702. It may be appreciated that the distance into the diseased tissue may vary based on parameter values, treatment times and type of tissue, to name a few. It may also be appreciated that larger or smaller treatment depths may be achieved than illustrated herein.

The delivered energy treats the diseased tissue D as appropriate. In the case of cancer, the cancerous cells are destroyed, eliminated, killed, removed, etc. In the PEF zone, such cell death occurs while maintaining non-cancerous, non-cellular elements, such as collagen, elastin, and matrix proteins. In some instances, these non-cellular elements maintain the structure of the tissue allowing for and encouraging normative cellular regeneration. Likewise, in some instances, when the energy reaches walls W of a nearby luminal structure LS the integrity and mechanical properties of the luminal structure LS is preserved. It may be appreciated that in some instances, the energy kills the cells in the diseased tissue D directly, such as via accumulated generalized cellular injury and irrecoverable disruption of cellular homeostasis. In other instances, the cells die by action of the immune system or other biological processes. In some instances, remaining diseased tissue is surgically removed or removed by other methods.

### A. Alternative Probe Designs

It may be appreciated that the probe 500 may have a variety of forms and structures. In some embodiments, the probe 500 is hollow, such as having a tubular shape. In such embodiments, the probe 500 may be formed from a hypotube or metal tube. Such tubes can be optimized for desired push and torque capabilities, kink performance, compression resistance and flexibility to ensure consistent and reliable steerability to the target treatment site. Likewise, such tubes can include custom engineered transitions, such as laser cutting and skive features, along with optional coatings to optimize produce performance. In some embodiments, the tube has a sharp point with multiple cutting edges to form the probe tip 502. In other embodiments, the tube has a blunt atraumatic tip. In some embodiments, the probe 500 is solid, such as having a rod shape. These probes can also be optimized and customized similarly to hypotubes. In some embodiments, the solid probe 500 has a sharp point with a symmetric or asymmetric cut to form the probe tip 502. In other embodiments, the solid probe 502 has a blunt atraumatic tip.

It may be appreciated that the probe 500 may include a lumen for delivery of fluids or agents. Such a lumen may be internal or external to the probe. Likewise, fluid or agents may be delivered directly from the shaft 106, such as through a lumen therein or a port located along the shaft 106.

In some embodiments, the probe 500 is comprised of multiple probe elements, wherein each probe element has similar features and functionality to an individual probe 500 as described above. Thus, in some embodiments they may be considered separate probes, however for simplicity they will be described as probe elements making up a single probe 500 since they are passed through the same shaft 106 of the instrument 102. It may be appreciated that any number of probe elements may be present, including one, two, three, four, five, six, seven, eight, nine, ten or more. Likewise, the probe elements may be extended the same or different distances from the shaft 106 and may have the same or different curvatures. In another embodiment, the probe elements to not have any curvature and exit from the shaft 106 in a linear fashion. Typically, the probe elements are pre-curved so that advancement of the probe tip from the shaft 106 allows the probe element to assume its pre-curved shape. Thus, in some embodiments, a variety of curvatures can be utilized by advancing the probe tips differing amounts from the shaft 106.

It may be appreciated that the size of the probe tip 502 capable of transmitting energy may be further adjusted with the use of an insulating sheath 552 that extends at least partially over the probe. As mentioned previously, the size of the active portion of the probe tip 502 may be adjusted based on its extension from the shaft 106. However, this may be further refined, particularly when a plurality of probe elements are present, with the use of insulating sheaths 552 covering portions of the individual probe elements.

It may be appreciated that any of the probe elements described herein may have the same structure and features as any of the probes describe herein. For example, the probe elements may be constructed of the same materials, have the same functionality and have a sharp or atraumatic tip. Likewise, it may be appreciated that any of the probe elements may be deployed independently or simultaneously and may be energized independently or simultaneously. The energy delivered may be provided by the same energy delivery algorithm 152 or different energy delivery algorithms 152, therefore delivering the same or different energies. Any of the probe elements may function in a monopolar manner or in a bipolar manner between pairs of probe elements. Likewise, it may be appreciated that the probe elements may function in a combination of monopolar and bipolar manners.

As stated previously, in many of these extra-luminal delivery embodiments, the energy delivery body 108 has the form of a probe 500 that is disposed within the lumen 105 of the shaft 106. In some embodiments, the probe 500 comprises a plurality of wires or ribbons 120 and forms a basket 555 serving as an electrode. It may be appreciated that alternatively the basket 555 can be laser cut from a tube. It may be appreciated that a variety of other designs may be used. Typically, the basket 555 is delivered to a targeted area in a collapsed configuration and then expanded for use.

### V. Imaging

Methods associated with imaging that can be useful include: (a) detecting diseased target tissue, (b) identifying areas to be treated, (c) assessing areas treated to determine how effective the energy delivery was, (d) assessing target areas to determine if areas were missed or insufficiently treated, (e) using pre- or intra-procedural imaging to measure a target treatment depth and using that depth to choose a specific energy delivery algorithm to achieve tissue effects to that depth, (f) using pre or intra-procedural imaging to identify a target cell type or cellular interface and using that location or depth to choose a specific energy delivery algorithm to achieve tissue effects to that target cell type or cellular interface, and/or (g) using pre-, intra-, or post-procedural imaging to identify the presence or absence of a pathogen with or without the presence of inflamed tissue.

In some embodiments, confocal laser endomicroscopy (CLE), optical coherence tomography (OCT), ultrasound, static or dynamic CT imaging, X-ray, magnetic resonance imaging (MRI), and/or other imaging modalities can be used, either as a separate apparatus/system, or incorporated/integrated (functionally and/or structurally) into the treatment system 100 by either incorporating into the instrument 102 or a separate device. The imaging modality (or modalities) can be used to locate and/or access various sections of target tissue. In some embodiments, the targeted depth of treatment can be measured and used to select a treatment algorithm 152 sufficient to treat to the targeted depth. At least one energy delivery body can then be deployed at the target tissue site and energy delivered to affect the target tissue. The imaging modality (or modalities) can be used before, during, between, and/or after treatments to determine where treatments have or have not been delivered or whether the energy adequately affected the airway wall. If it is determined that an area was missed or that an area was not adequately affected, the energy delivery can be repeated followed by imaging modality (or modalities) until adequate treatment is achieved. Further, the imaging information can be utilized to determine if specific cell types and or a desired depth of therapy was applied. This can allow for customization of the energy delivery algorithm for treating a wide variety of patient anatomies.

In some embodiments, access via a body lumen is visualized with one or more appliances inserted into the body. Likewise, in some embodiments, one or more of a variety of imaging modalities (e.g., CLE, OCT) are used either along with direct visualization, or instead of direct visualization. As an example, a bronchoscope can be delivered via the mouth to allow for direct visualization and delivery of the instrument 102, while an alternate imaging modality can be delivered via another working channel of the bronchoscope, via the nose, or adjacent to the bronchoscope via the mouth. In some embodiments, the imaging modality (e.g., direct visualization, CLE, and/or OCT) is incorporated into the instrument 102 with appropriate mechanisms to connect the imaging modality to either the system generator 104 or commercially available consoles.

### VI. Conditioning

It may be appreciated that although the PEF ablation treatments provided by the systems 100 may be used as conditioning for other treatments, the target tissue cells may alternatively be conditioned prior to the PEF ablation treatments provided by the systems 100.

In some embodiments, cells targeted for treatment are conditioned so as to modify the behavior of the cells in response to the delivery of the energy signals. Such conditioning may occur prior to, during, or after delivery of the energy signals. In some embodiments, conditioning prior to energy delivery is considered pre-conditioning and conditioning after energy delivery is considered post-conditioning. Such differentiation is simply based on timing rather than on how the conditioning treatment affects the cells. In other embodiments, pre-conditioning relates to affecting what happens to the cells during energy delivery, such as how the cells uptake the energy, and post-conditioning relates to affecting what happens to the cells after energy delivery, such as how the cells behave after receiving the energy. Such differentiation may be less relevant to timing since in some instances conditioning may occur prior to energy delivery but only affect the cellular response following the energy delivery. Therefore, it may be appreciated that “conditioning” may be considered to apply to each of these situations unless otherwise noted.

Typically, conditioning is achieved by delivering a conditioning solution. In the case of intra-luminal therapy, the conditioning solution may be delivered via the luminal structure. The conditioning solution may alternatively or additionally be delivered via direct fluid injection of the conditioning solution into the targeted region, either from an endoluminal or other approach. In some embodiments, the conditioning solution selectively alters the electrical properties of the target cells, such as to affect the way the pulsed energy delivery gets distributed. In other embodiments, the conditioning solution influences the activity of the target cells. For example, in the lung such conditioning solution may promote basal cell differentiation into ciliated cells and/or downregulate goblet cells and submucosal gland cells. In other embodiments, the conditioning solution increases the likelihood of the target cells to expire following pulsed energy delivery. In still other embodiments, the conditioning solution alters the responses of non-targeted cells to the pulsed electric fields. In alternate embodiments, conditioning is performed via non-solution-based exposure of the tissues. This includes radiation therapy, radiotherapy, proton beam therapy, etc. In some embodiments, the conditioning will impact the enzymatic and energy-producing components of the cellular infrastructure.

The conditioning solution may be comprised of a variety of agents, such as drugs, genetic material, bioactive compounds, and antimicrobials, to name a few. For embodiments where the conditioning solution increases the likelihood of the target cells to expire following pulsed energy delivery, the conditioning solution may comprise chemotherapy drugs (e.g. cisplatin, doxorubicin, paclitaxel, bleomycin, carboplatin, etc), calcium, antibiotics, or toxins, to name a few. For embodiments where the conditioning solution alters the responses from non-targeted cells to the pulsed electric fields, the conditioning solution may comprise cytokines (e.g. immunostimulants, such as interleukins), genes, VEGF (e.g. to encourage more vessel growth into area), cellular differentiating factors (e.g. molecules to promote conversion of goblet cells into ciliated cells), and/or other small molecules that interact with cells, such as agonists and antagonists for receptors such as programmed death (PD-1) or programmed death ligand (PD-L1). Conditioning solutions may be delivered at the targeted site to directly interact at the site of PEF energy delivery. They may also be delivered systemically (e.g., intravenously, intraperitoneally) or regionally (e.g., intravenously to feeding arterial supplies for the targeted region, higher feeding airway generations, intraparenchymally to tissue around the tissue directly affected by the PEF treatment).

In some embodiments, the conditioning solution includes cells, such as stem cells, autograft cells, allograft cells or other cell types. In these embodiments, the cells may be used to alter the tissue response to the pulsed electric fields. In other embodiments, the cells may be used to repopulate the affected area with healthy or desirable cells. For example, once target cells have been weakened or killed by the delivered pulsed energy treatment, the cells from the conditioning solution may move into the vacancies, such as a decellularized extracellular matrix. In some embodiments, the area is washed out to remove the dead cells, such as with a mild detergent, surfactant or other solution, prior to delivery of the conditioning solution containing the new cells. In other embodiments, mechanical stimulation, such as suction, debriding, or ultrasonic hydrodissection, is used to physically remove the dead cells prior to delivery of the conditioning solution containing the new cells.

In some embodiments, the conditioning provided may invoke a targeted immune response. The immune response may result in a number of factors that alter the treatment effect outcome. This may result in an increase in the systemic immunity upregulation using specific markers associated with some targeted tissue, such as a tumor or bacteria or virus associated with an infection. It may also result in an upregulation of the innate immunity that broadly affects the immune system functionality to detect general abnormal cells, bacteria, or other infectious organisms residing within the body, which may occur locally, regionally, or systemically.

In some embodiments, the conditioning solution is warmed or chilled to alter how the target cells respond. Generally, warmed solutions promote increased treatment effects (e.g. increased susceptibility to cell death), while chilled solutions would reduce the extent of treatment effect or increase cell survival after exposure to a reversibly-designed protocol. In some embodiments, a chilled conditioning solution comprised of genes and or drugs is used to precondition cells to survive energy delivery treatment, increasing the number of cells that survive the treatment. In some embodiments, the effects of the warmed/chilled conditioning solution is compounded with the general effects caused by the other agents in the solution (e.g. warmed calcium solution, chilled gene containing solution). In other embodiments, the warmed/chilled conditioning solution does not provide effects other than temperature changes. In such embodiments, the conditioning solution is typically comprised of isotonic saline, phosphate buffered solution or other benign solution.

It may be appreciated that such heating or cooling may alternatively be achieved by other methods that do not involve delivery of a conditioning solution. For example, the target tissue may be heated or cooled by contacting the tissue with a warmed/cooled device, deliberately warming/cooling the pulsed electric field delivery catheter, delivering mild cryotherapy, or delivering mild radiofrequency or microwave energy. As previously described, this could promote enhanced lethality or permeability effects to the tissue or it could provide protective aspects to the cells that enable them to survive the procedure and exude the desired change as was targeted for them as a result of the therapy.

In some embodiments, a conditioning solution is delivered systemically, such as by intravenous injection, ingestion or other systemic methods. In other embodiments, the conditioning solution is delivered locally in the area of the targeted cells, such as through a delivery device or the instrument 102 itself.

### VII. Energy Delivery Algorithms and Lesion Optimization

The specialized energy is provided by one or more energy delivery algorithms 152. In some embodiments, the algorithm 152 prescribes a signal having a waveform comprising a series of energy packets wherein each energy packet comprises a series of high voltage pulses. In such embodiments, the algorithm 152 specifies parameters of the signal such as energy amplitude (e.g., voltage) and duration of applied energy, which is comprised of the number of packets, number of pulses within a packet, the fundamental frequency of the pulse sequence, duration of the individual pulses or sequence of pulses comprising a packet (which may change within the packet itself) to name a few. Additional parameters may include switch time between polarities in biphasic pulses, dead time or inter-cycle delay between biphasic cycles, and rest time or inter-packet delay between packets, which will be described in more detail in later sections. In some embodiments, there is a fixed inter-packet delay between packets. In some embodiments, packets are gated to the cardiac cycle and are thus variable with the patient's heart rate or are fixed so as to and synchronized with the cardiac cycle. In some embodiments, there is a deliberate, varying inter-packet delay algorithm or no rest period may also be applied between packets. Packet delivery may also be coordinated with multiple factors, such as a minimum inter-packet delay after which a next trigger signal (e.g., cardiac synchronization signal) is used to coordinate the timing of the subsequent packet delivery. It may be appreciated that a feedback loop based on sensor information and an auto-shutoff specification, and/or the like, may be included.

The specialized energy delivered to the target tissue by a probe 702 or other energy delivery body creates a lesion that has a plurality of zones. The zones emanate from the probe 702 radially outwardly, such as in rings. Each zone has differing cellular effects and therefore differing effects on the overall outcome of the treatment. In some instances, the zones include a cavitation zone, a thermal zone, a PEF zone, an inflammatory zone and an immune response zone.

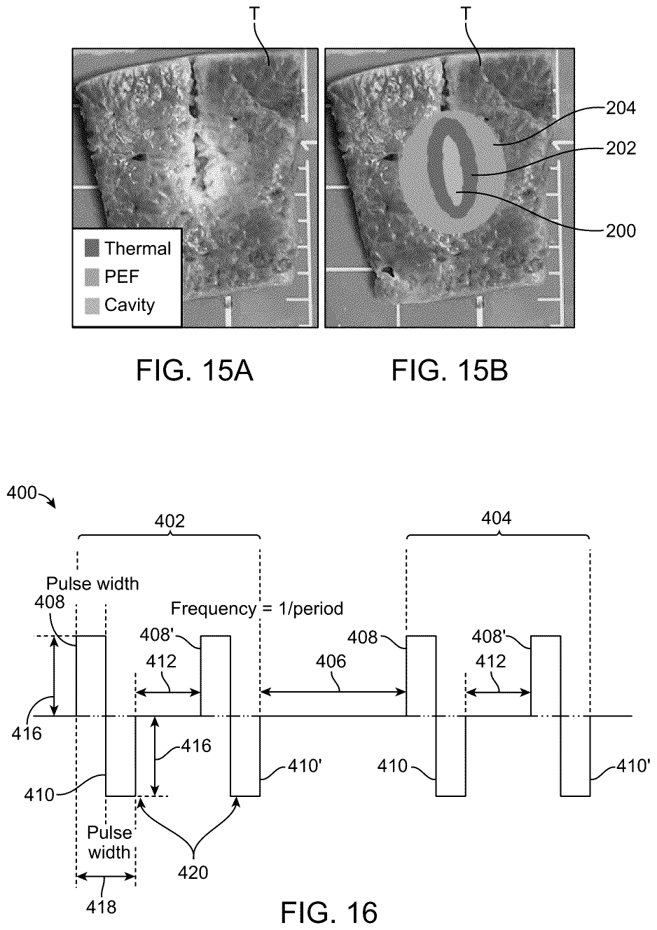

FIG. 15A illustrates an actual tissue lesion in tissue T created by delivering specialized energy to a porcine liver kept viable using a machine perfused organ preservation model. FIG. 15B illustrates the lesion demarcated with zones for clarity. The energy waveform generated a very small cavitation zone 200 (in this example, likely not an actual cavity but a needle tract from positioning the energy delivery body (needle) therein, however a cavitation zone 200 would typically be located here), a thermal zone 202, and a PEF zone 204. It may be appreciated that an inflammatory zone and/or immune response zone would be located around the PEF zone, thus following the edge of the PEF zone. The presence and size of the zones may be manipulated by the waveform, particularly the parameter values. It may be appreciated that in these examples a lesion is generated by a single dose or a single application of energy. The on-time of energy and its application during this dose is dependent on the waveform, more particularly the waveform parameters.

Energy delivery may be actuated by a variety of mechanisms, such as with the use of an actuator 132 on the instrument 102 or a foot switch operatively connected to the generator 104. Such actuation typically provides a single energy dose. The energy dose is defined by the number of packets delivered and the voltage of the packets. Each energy dose delivered to the target tissue maintains the temperature at or in the target tissue so as to generate the desired zones within the lesion. Zones such as PEF zones are maintained below a threshold for thermal ablation, particularly thermal ablation or denaturing of stromal proteins. In addition, the doses may be titrated or moderated over time so as to further reduce or eliminate thermal build up during the treatment procedure. Instead of inducing thermal damage (defined as protein coagulation at sites of danger to therapy) in PEF zones, the energy dose provides energy at a level which induces treatment of the condition, such as cancer, without damaging sensitive tissues.

FIG. 16 illustrates an embodiment of a waveform 400 of a signal prescribed by an energy delivery algorithm 152. Here, two packets are shown, a first packet 402 and a second packet 404, wherein the packets 402, 404 are separated by a rest period or interpacket-delay 406. In this embodiment, each packet 402, 404 is comprised of a first biphasic cycle (comprising a first positive pulse peak 408 and a first negative pulse peak 410) and a second biphasic cycle (comprising a second positive pulse peak 408′ and a second negative pulse peak 410′). The first and second biphasic pulses are separated by dead time or inter-cycle delay 412 (i.e., a pause) between each pulse. In this embodiment, the biphasic pulses are symmetric so that the set voltage 416 is the same for the positive and negative peaks. Here, the biphasic, symmetric waves are also square waves such that the magnitude and time of the positive voltage wave is approximately equal to the magnitude and time of the negative voltage wave.

It may be appreciated that manipulation of the various parameters (e.g. voltage, fundamental frequency, number of pulses per packet, number of packets, and various delays, etc.) have a variety of influences on the resultant lesion and on the body itself. In some instances, parameter changes can balance each other wherein the effect of change in one or more parameters can be balanced by a change in one or more different parameter values leading to the same or similar result. In other instances, parameter values can be tuned to create or generate different effects, such as different lesion characteristics (e.g. the presence, size and/or properties of the different zones) and/or different effects on the body. These effects on the body may be immediate, such as muscle stimulation, or delayed, such as the generation of a particular immune response.

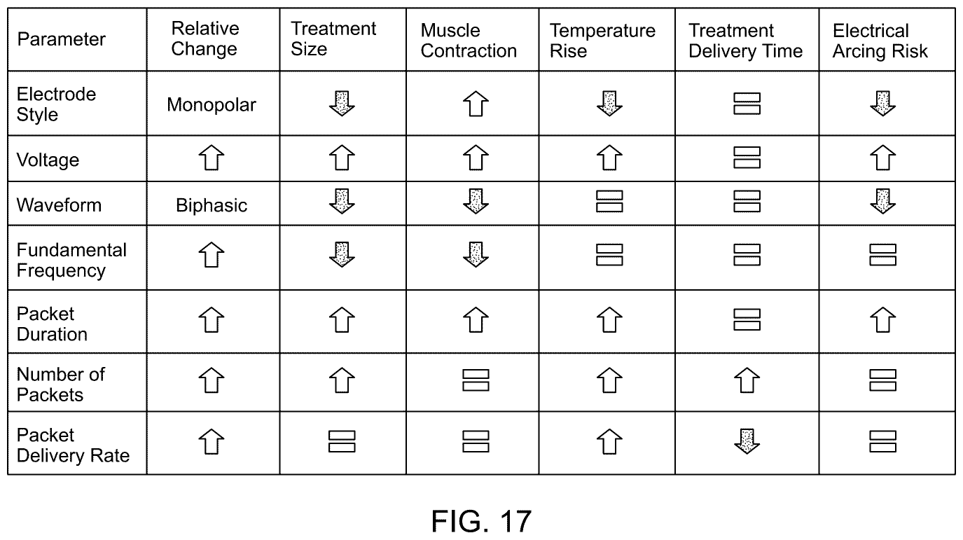

FIG. 17 provides a table illustrating various example effects of parameter changes. Typically, the electrode style is monopolar as opposed to bipolar. In monopolar arrangements, one or more delivery electrodes are positioned near the target tissue site and at least one remote return electrode is positioned against the patient's skin. By utilizing a monopolar electrode configuration, muscle contraction intensity increases. To counteract this effect, generally a biphasic waveform or waveform comprised of sufficiently short individual pulse durations comprising a packet with appropriate delays between the pulses, so as to offset the degree in muscle contraction. This waveform variety results in treatment effect decreases, but the decrease is more subtle than the muscle contraction reduction, thus resulting in still valid PEF therapeutic applications. Monopolar configurations also decrease the risk of electrical arcing compared to bipolar or multipolar configurations, wherein all effector electrodes are placed within a similar region that may permit electrical arcing between them. Typically, the waveform utilizes biphasic pulses as opposed to monophasic pulses. The use of biphasic pulses decreases the treatment size, decreases muscle contraction and also decreases arcing risk. Thus, the use of biphasic pulses counters the increase in muscle contraction due to the monopolar electrode style. Since both monopolar electrode style and biphasic pulses reduce treatment size, treatment size can be increased by changing other variables. For example, an increase in voltage, packet duration and number of packets increases the treatment size. However, increasing these parameters have a variety of other effects. For example, increasing the voltage and increasing the packet duration each increase muscle contraction, temperature rise and risk of electrical arcing. Increasing the fundamental frequency can lower the muscle contraction but it also reduces the treatment size. Likewise, increasing the number of packets increases the treatment size but also increases the temperature rise and the treatment delivery time. The treatment delivery time can be lowered by increasing the packet delivery rate, however, this increases the temperature rise. Therefore, managing the influences of various parameter changes is a complex endeavor. This is further complicated by the increments in which these changes are made. Once considering the number of parameters and increments in which they can be changed as related to the PEF waveform and dose itself, the set of combinations is staggering in volume. This is further compounded by the influence of electrode geometry, such as where a large electrode delivering a particular electrical voltage will have different characteristics than a smaller electrode, or monopolar versus bipolar and multipolar arrangements (and the separation distances between electrodes in these arrangements). These characteristics also include the temperature rise, treatment effect size, electrical current delivered, muscle contraction, electrical arcing risk, and time required to attain coverage of a targeted treatment effect size. Furthermore, certain conditioning solutions provided to the patient may specifically be used to target and reduce the induced muscle contraction. For instance, neuromuscular blockade with pancuronium bromide, vecuronium, succynlcholine, and other blockades may be used. This reduction in muscle contraction may be used to facilitate treatment doses with lower frequencies, longer packet durations, or higher voltages to attain larger treatment effects while maintaining acceptably safe muscle contractions.

Thus, determining the appropriate parameter values for a particular outcome involves a high level of manipulation and skill. In addition, determining what particular outcome is desired also involves a high level of skill. For example, the presence, type and size of the various zones in a resultant lesion that culminates in a desired clinical outcome has not been previously known. The methods, systems and devices described herein provide both the specific parameter values and the characteristics of the desired resultant lesions that culminate in the desired clinical outcomes. Thus, the specialized energy delivered to the target tissue, typically cancerous or otherwise undesired, is generated from an algorithm 152 of the generator 104 that produces a waveform according to these parameter values.

In one embodiment, the treatment dose is provided by a waveform that is produced from a combination of parameter values that includes the following: voltage=3000V, fundamental frequency=400 kHz, number of biphasic pulses (i.e., cycles) per packet=40 cycles, inter-cycle delay=1000 microseconds, number of packets=100 packets and inter-packet delay=3 seconds. For this combination, the dose delivery time is approximately 5-6 minutes. This treatment time is substantially shorter than treatment times using conventional energy such as microwave (10 minutes), RF (20 minutes), or cryoablation (30-40 minutes). In some instances, this dose produces a lesion size of approximately 1×1×1 cm³±0.3 cm in any direction.

In addition to manipulating the parameter values to generate desired lesion zones, parameter values were further manipulated to maximize lesion size while ensuring patient safety, a desirable treatment time, reduced electrical arcing risk, reduced muscle contraction, and reduced temperature rise, to name a few. Extensive studies were undertaken to determine the parameters for the desired dose. Studies included evaluating the following parameter combinations:

TABLE 1

| | | Fundamental | | Inter- | | | |
|---|---|---|---|---|---|---|---|
| | Voltage, | Frequency, | Cycles, | Cycle | | Packets, | Inter-Packet |
| No. | V | kHz | count | Delay, μs | | count | Delay, s |
| 1 | 2500 V | 400 kHz | 40 cycles | 1000 | μs | 100 | 3 second |
| | | | | | | packets | |
| 2 | 2500 V | 400 kHz | 60 cycles | 1000 | μs | 100 | 3 second |
| | | | | | | packets | |
| 3 | 3000 V | 400 kHz | 40 cycles | 1000 | μs | 100 | 3 second |
| | | | | | | packets | |
| 4 | 3000 V | 400 kHz | 60 cycles | 1000 | μs | 100 | 3 second |
| | | | | | | packets | |
| 5 | 3300 V | 400 kHz | 60 cycles | 1000 | μs | 100 | 3 second |
| | | | | | | packets | |
| 6 | 2500 V | 400 kHz | 40 cycles | 500 | μs | 100 | 3 second |
| | | | | | | packets | |
| 7 | 2500 V | 400 kHz | 60 cycles | 500 | μs | 100 | 3 second |
| | | | | | | packets | |
| 8 | 3000 V | 400 kHz | 40 cycles | 500 | μs | 100 | 3 second |
| | | | | | | packets | |
| 9 | 3000 V | 400 kHz | 60 cycles | 500 | μs | 100 | 3 second |
| | | | | | | packets | |
| 10 | 3300 V | 400 kHz | 60 cycles | 500 | μs | 100 | 3 second |

packets

Thus, the refined dose of specialized energy described herein generates a treatment lesion (e.g. 10 mm in diameter) with minimal thermal volume, particularly in comparison to lesions generated by conventional pulsed electric field technology. In some embodiments, the treatment duration is 5 minutes. The refined dose provided by the specialized waveforms maximizes the PEF treatment effect zone while minimizing thermal effects. At this specifically-tuned dosing, the PEF energy stimulates the body's innate and adaptive immune response which is described in more detail herein below.

### VIII. Immune Response

The specialized PEF energy described herein orchestrates activation of multiple aspects of the host immune response which cause cell death, including antibody-dependent cell-mediated cytotoxicity (ADCC). ADCC is a mechanism of cell-mediated immune defense whereby an effector cell of the immune system kills a target cell, whose membrane-surface antigens have been bound by specific antibodies. Such target cells include tumor cells.

Immunoglobulin M (IgM) is the first antibody the body makes when it fights a new infection. IgM is found mainly in blood and lymph fluid. Following the production of IgM antibodies, there is a subsequent production of immunoglobulin G (IgG) which is the most common antibody. It is found in blood and other body fluids and protects against bacterial and viral infections. IgG can take time to form after an infection or immunization. However, it is ultimately the most abundant immunoglobulin in the bloodstream. IgG antibodies are smaller and more versatile than IgM antibodies. They are responsible for long-term immunity and play a crucial role in neutralizing pathogens and enhancing the immune response. While IgM is the first antibody to be produced after vaccination, IgG production follows and is responsible for the sustained and long-term immune response.

It may be appreciated that immunoglobin E (IgE) also plays a role. While the predominant functions of IgE are related to allergies, IgE also plays a role in anti-tumor immune responses. In the context of anti-tumor immunity, the role of IgM is less well-defined compared to IgG or IgE. However, there are some potential roles of IgM in anti-tumor immune responses.

ADCC is one of the mechanisms through which antibodies, such as these described herein, can act to limit and contain infection as part of the humoral immune response. Likewise, through this mechanism, antibodies can also kill tumor cells. In general, ADCC has typically been described as the immune response to antibody-coated cells leading ultimately to the lysing of the infected or diseased cell.

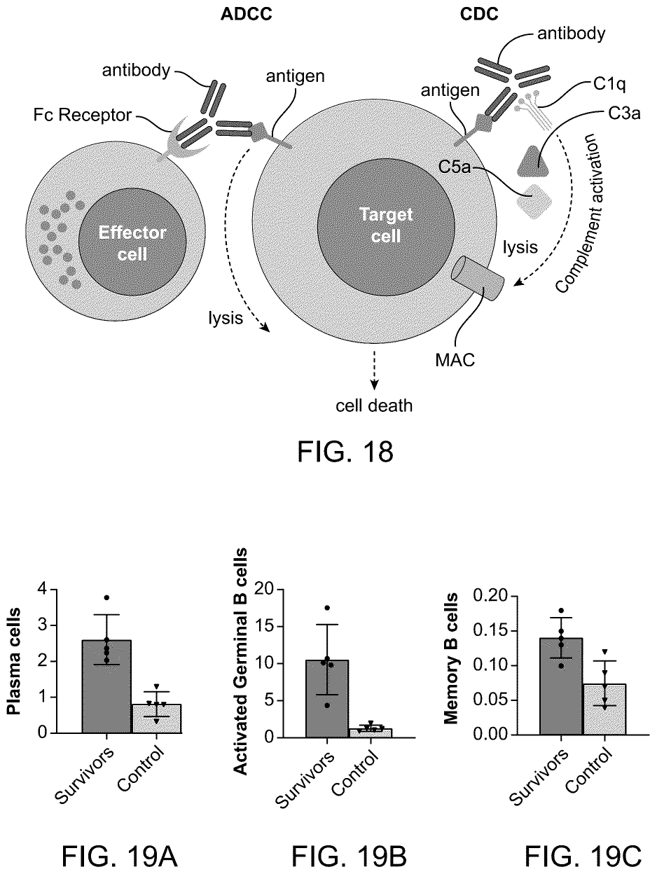

Referring to FIG. 18, ADCC involves activation of natural killer cells by antibodies in a multi-tiered progression of immune control. A natural killer cell expresses crystallizable fragment (Fc) receptors. These receptors recognize and bind to the reciprocal portion of an antibody, such as immunoglobulin G (IgG), which binds to the surface of a target cell, such as a tumor cell. The most common of these Fc receptors on the surface of a natural killer cell is CD16 or FcγRIII. Once the Fc receptor binds to the Fc region of the antibody, the natural killer cell releases cytotoxic substances, such as perforin and granzymes, that directly induce cell death in tumor cells. Thus, ADCC is independent of the immune complement system that also lyses targets but does not require any other cell. ADCC involves an effector cell which classically is known to be natural killer cells that typically interact with IgG antibodies. However, macrophages, neutrophils and eosinophils can also mediate ADCC. NK cells and macrophages can also engulf and destroy tumor cells through a process called phagocytosis. IgG antibodies can coat tumor cells through a process called opsonization. This coating makes tumor cells more recognizable and susceptible to engulfment by phagocytic cells, such as macrophages. Phagocytosis of opsonized tumor cells facilitates their clearance and elimination by the immune system.

It may be appreciated that IgG antibodies can also activate the complement system, which is a part of the immune system. Activation of the complement system leads to the formation of a membrane attack complex that creates pores in the cancer cell membrane, causing it to rupture and leading to cell death. Thus, this is called Complement-Dependent Cytotoxicity (CDC), also illustrated in FIG. 18.

The specialized PEF energy described herein is involved in these various antibody related cell death mechanisms leading to tumor cell death. This is evidenced by a variety of examples. In one example, laboratory mice were provided, each having three tumors. Two of the three tumors were treated with the specialized PEF energy. Consequently, the two treated tumors disappeared due to direct treatment by the PEF energy and the third untreated tumor also disappeared due to an abscopal effect. Six months later, the mice were inoculated with tumor cells and the mice rejected the tumors. Fifteen months later, the mice were again inoculated with tumor cells and again the mice rejected the tumors. One month later, the mice were again inoculated with tumor cells (a third rechallenge) and again the mice rejected the tumors. Fourteen days later, the mice were euthanized to collect various organs for flow cytometry analysis. Select results are illustrated in FIGS. 19A-19C which illustrate an increase in plasma cells, activated germinal B cells and memory B cells in the mice that received the specialized PEF energy. This indicates that antibody production increased due to the treatment by the specialized PEF energy. This antibody is most likely IgG but could also include IgM and IgE. The flow cytometry analysis did not show significant changes in T-cells between the treated mice and the control groups.

In some embodiments, the specialized PEF energy is delivered to a target treatment area, such as including a tumor, in a manner that promotes the upregulation of PD-L1 by the tumor cells. In this embodiment, the tumor T becomes stressed due to the inflammatory insult induced by the PEF energy which transcriptionally activates PD-L1. It may be appreciated that activation of PD-L1 may typically be considered detrimental in the treatment of tumors in that such upregulation of PD-L1 increases the immunosuppressive interaction of PD-1/PD-L1 thereby inhibiting T cells (e.g. CD8+ T cells). However, such upregulation has the capability of making cold tumors (i.e. a tumor that is PD-L1 negative and has a low tumor immune cell infiltrate) PD-L1 positive and thereby hot. A hot tumor is PD-L1 positive and has a high tumor immune cell infiltrate. Patients that have hot tumors will be responsive to checkpoint therapy targeting the PD-1/PD-L1 interaction. Thus, patients that have cold tumors, either originally or by developed resistance, can be transformed into having hot tumors with appropriate treatment by PEF energy.

Thus, in some embodiments, an antibody targeting PD-1 (αPD-1) is delivered to the patient, either in combination with the PEF energy or at a predetermined time or times in relation to the PEF energy. The αPD-1 interferes with the PD-1/PD-L1 interaction, allowing the tumor cells to be identified and killed by the T cells. Thus, the transformed patient is now responsive to immunotherapy such as αPD-1 therapy.

The effects of the PEF therapy and checkpoint inhibition on tumor growth rate, survival, and the expression of pro/anti-inflammatory cytokines have been studied in two syngeneic models of mammary carcinoma: EMT6 and 4T-1 tumor bearing Balb/c mice. Since murine models of cancer have varying levels of immunogenicity (i.e., the ability of the transplanted cancer to invoke an immune response), it was desired to validate therapy across multiple tumor models to accurately determine immunologic outcomes.

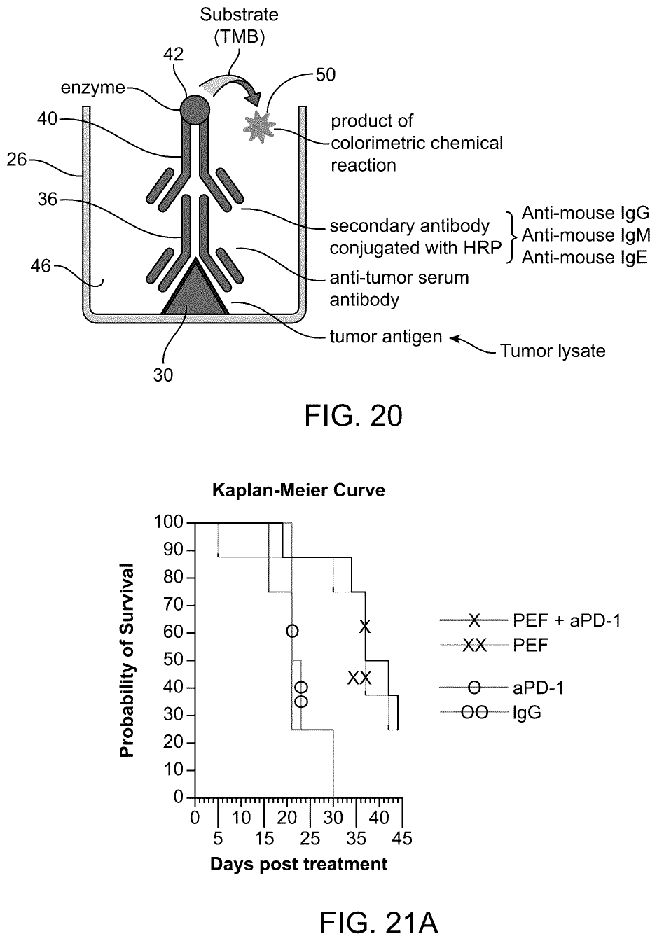

In the EMT6 tumor model, a combinatorial therapeutic protocol was undertaken that included both specialized PEF therapy and checkpoint inhibition (delivery of αPD-1), known as “PEF+αPD-1”. This was compared to PEF delivery alone and αPD-1 alone. In this tumor model, each female mouse was challenged with EMT6 cells in the 4th/5th mammary fat (left side of the animal). Twelve days after the challenge, tumors were directly treated with PEF energy. In some instances, the mouse was administered αPD-1 once a week starting on either the day of initial challenge with EMT6 cells or on the day of specialized PEF energy delivery. In other instances, the mouse was administered αPD-1 multiple times a week starting on the day of initial challenge with EMT6 cells or on the day of PEF energy delivery. Three months after PEF treatment, all animals were challenged with EMT6 cells in the contralateral side (4th/5th mammary fat on the right side of the animal) and all animals rejected the tumor. Six months later, serological analysis was undertaken. FIG. 20 illustrates a schematic of a custom ELISA workflow for the serological analysis as described previously in the above described methods (e.g. FIGS. 1A-1C, FIGS. 2A-2B, 3A-3C, FIG. 4, FIGS. 5A-5B, FIGS. 6-8). Antibodies 36 are “captured” by a reagent containing secondary antibodies 40 that is involved in a chemical reaction that transforms a clear liquid into a blue liquid according to the quantity of captured antibodies 36. A plate reader then reads the intensity of the blue color. The more blue color, the higher the optical density (OD) and therefore the higher the amount of detected antibodies 36.

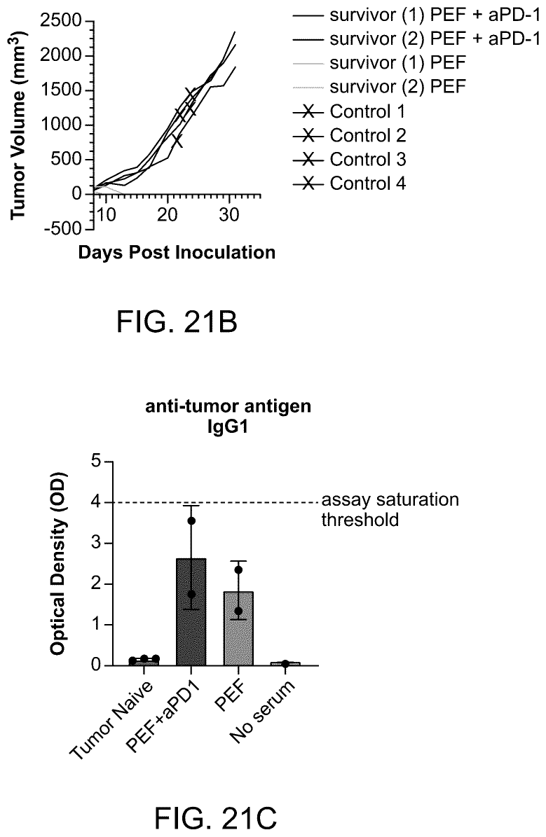

The results of the study are shown in FIGS. 21A-21C. As shown, the treatment with specialized PEF stimulates the production of antibodies (e.g. IgG), similarly to a vaccination, that persist in blood for an extended period of time.

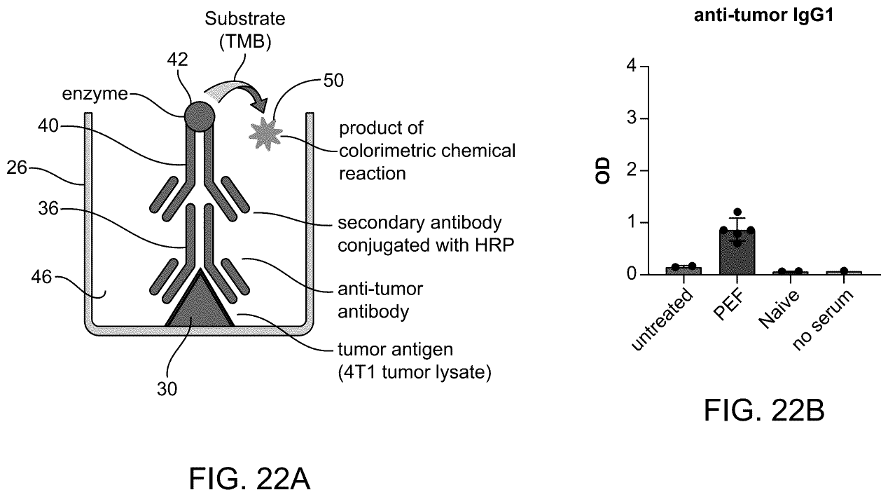

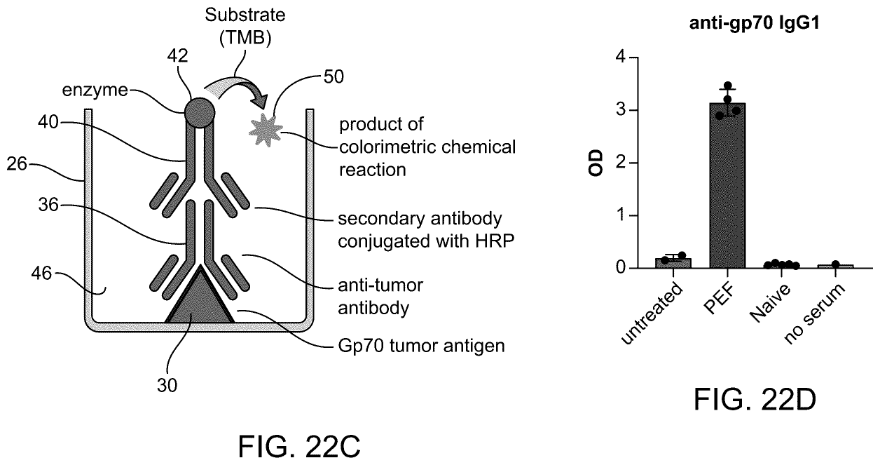

In the 4T-1 tumor model, a more aggressive, metastatic tumor model, a PEF therapeutic protocol was also utilized. Results compared specialized PEF delivery alone to untreated mice and naïve mice. Serum was collected 20 days after ablation with the specialized PEF energy. Blood was also analyzed from untreated mice at the same time point. The results are shown in FIGS. 22A-22D which indicate that the treatment with specialized PEF energy promotes more antibody production. FIGS. 22A-22B quantify antibodies 36 that are targeting a pool of many tumor antigens 30. FIGS. 22C-22D quantify antibodies 36 that are specific to one tumor antigen, gp70. Both gave similar results indicating that the assay is reliable.

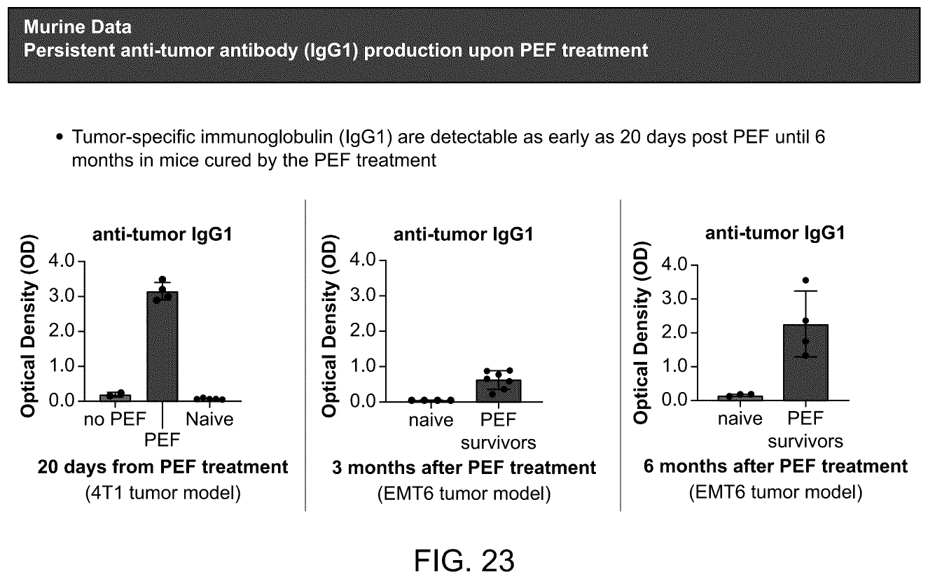

FIG. 23 summarizes IgG data from the 4T-1 tumor model and the EMT6 tumor models described above, with the addition of IgG data from an EMT6 tumor model wherein blood was analyzed three months after treatment with specialized PEF energy. This summary illustrates that tumor specific immunoglobin (IgG1) is detectable from at least as early as 20 days after specialized PEF treatment and at least up to 6 months after specialized PEF treatment. It may be appreciated that the data analyzed three months after treatment shows a lower optical density because the mice were not rechallenged, and thus did not have a “boost” from rechallenging, similar to a booster vaccine.

Thus, when treating cancerous tumors, treatment with specialized PEF energy results in an increased production of antibodies. This increases improves survival as the treatment harnesses the body to further eliminate cancerous cells such as residual cancer cells, metastatic cancer cells or later developing cancer cells.

### Altered PEF Dosing

As stated previously, the specialized treatment dose is provided by a waveform that is produced from a combination of parameter values that includes the following: voltage=3000V, fundamental frequency=400 kHz, number of biphasic pulses (i.e., cycles) per packet=40 cycles, inter-cycle delay=1000 microseconds, number of packets=100 packets and inter-packet delay=3 seconds. It may be appreciated that deviations from this specialized dose typically generate different results.

Nonetheless, it may be appreciated that different parameters may be used, typically with different outcomes. The different outcomes may be different in some aspects and similar in other aspects. Likewise, the different outcomes may be preferred in some situations and not in others. The following further describe the parameters of the energy waveform:

### A. Voltage

The voltages used and considered may be the tops of square-waveforms, may be the peaks in sinusoidal or sawtooth waveforms, or may be the RMS voltage of sinusoidal or sawtooth waveforms. In some embodiments, the energy is delivered in a monopolar fashion and each high voltage pulse or the set voltage 416 is between about 500 V to 10,000 V, particularly about 3000V to 3300V, 3000V to 3500V, 3300 to 3500V, 3500 V to 4000 V, about 3500 V to 5000 V, about 3500 V to 6000 V, including all values and subranges in between including about 3000 V, 3300V, 3500 V, 4000 V, 4500 V, 5000 V, 5500 V, 6000 V to name a few. Voltages delivered to the tissue may be based on the setpoint on the generator 104 while either taking in to account the electrical losses along the length of the instrument 102 due to inherent impedance of the instrument 102 or not taking in to account the losses along the length, i.e., delivered voltages can be measured at the generator or at the tip of the instrument.

It may be appreciated that the set voltage 416 may vary depending on whether the energy is delivered in a monopolar or bipolar fashion. In bipolar delivery, a lower voltage may be used due to the smaller, more directed electric field. The bipolar voltage selected for use in therapy is dependent on the separation distance of the electrodes, whereas the monopolar electrode configurations that use one or more distant dispersive pad electrodes may be delivered with less consideration for exact placement of the catheter electrode and dispersive electrode placed on the body. In monopolar electrode embodiments, larger voltages are typically used due to the dispersive behavior of the delivered energy through the body to reach the dispersive electrode, on the order of 10 cm to 100 cm effective separation distance. Conversely, in bipolar electrode configurations, the relatively close active regions of the electrodes, on the order of 0.5 mm to 10 cm, including 1 mm to 1 cm, results in a greater influence on electrical energy concentration and effective dose delivered to the tissue from the separation distance. For instance, if the targeted voltage-to-distance ratio is 3000 V/cm to evoke the desired clinical effect at a desired tissue depth (1.3 mm), if the separation distance is changed from 1 mm to 1.2 mm, this would result in a necessary increase in treatment voltage from 300 to about 360 V, a change of 20%.

### B. Frequency

It may be appreciated that the number of biphasic cycles per second of time is the fundamental frequency when a signal is continuous. Since the specialized PEF waveforms are not continuous throughout the dose (e.g. the waveform includes packets and delays), the fundamental frequency is used as a method of describing the pulse width of the biphasic pulses of the specialized PEF waveform. It may be appreciated that the pulse width is the measure of the elapsed time between the leading and trailing edges of a single biphasic cycle and can be derived from a value of a fundamental frequency. For example, a waveform having a fundamental frequency of 400 Hz has a biphasic pulse width of 2.5 microseconds.

In some embodiments, biphasic pulses are utilized to reduce undesired muscle stimulation, particularly cardiac muscle stimulation. In other embodiments, the pulse waveform is monophasic and there is no clear inherent frequency. Instead, a fundamental frequency may be considered by doubling the monophasic pulse length to derive the frequency. In some embodiments, the signal has a frequency in the range 100 kHz-1 MHz, more particularly 100 kHz-1000 kHz, including 100 kHz, 200 kHz, 300 kHz, 400 kHz, 500 kHz, 100-500 kHz, etc. In some embodiments, the signal has a frequency in the range of approximately 100-600 kHz which typically penetrates a lumen wall so as to treat or affect particular cells somewhat deeply positioned, such as submucosal cells or smooth muscle cells. In some embodiments, the signal has a frequency in range of approximately 600 kHz-1000 kHz or 600 kHz-1 MHz which typically penetrates a lumen wall so as to treat or affect particular cells somewhat shallowly, such as epithelial or endothelial cells. It may be appreciated that at some voltages, frequencies at or below 100-250 kHz may cause undesired muscle stimulation. Therefore, in some embodiments, the signal has a frequency in the range of 400-800 kHz or 500-800 kHz, such as 400 kHz, 450 kHz, 500 kHz, 550 kHz, 600 kHz, 650 kHz, 700 kHz, 750 kHz, 800 kHz. In particular, in some embodiments, the signal has a frequency of 600 kHz. In addition, cardiac synchronization is typically utilized to reduce or avoid undesired cardiac muscle stimulation during sensitive rhythm periods. It may be appreciated that even higher frequencies may be used with components which minimize signal artifacts.

### C. Voltage-Frequency Balancing

The frequency of the waveform delivered may vary relative to the treatment voltage in synchrony to retain adequate treatment effect. Such synergistic changes would include the decrease in frequency, which evokes a stronger effect, combined with a decrease in voltage, which evokes a weaker effect. For instance, in some cases the treatment may be delivered using 3000 V in a monopolar fashion with a waveform frequency of 800 kHz, while in other cases the treatment may be delivered using 2000 V with a waveform frequency of 400 kHz.

When used in opposing directions, the treatment parameters may be manipulated in a way that makes it too effective, which may increase muscle contraction likelihood or risk effects to undesirable tissues, such as cartilage for airway treatments. For instance, if the frequency is increased and the voltage is decreased, such as the use of 2000 V at 800 kHz, the treatment may not have sufficient clinical therapeutic benefit. Opposingly, if the voltage was increased to 3000 V and frequency decreased to 400 kHz, there may be undesirable treatment effect extent to collateral sensitive tissues. In some cases, the over-treatment of these undesired tissues could result in morbidity or safety concerns for the patient, such as destruction of cartilaginous tissue in the airways sufficient to cause airway collapse, or destruction of smooth muscle in the GI tract sufficient to cause interruption of normal peristaltic motion. In other cases, the overtreatment of the untargeted or undesirable tissues may have benign clinical outcomes and not affect patient response or morbidity if they are overtreated.

### D. Cycles and Packets

As mentioned, the algorithm 152 prescribes a signal having a waveform comprising a series of energy packets wherein each energy packet comprises a series of high voltage pulses. Typically, the pulses are biphasic so they are referred to as cycles. Referring to FIG. 16, the first packet 402 has a cycle count 420 of two. In some embodiments, the cycle count 420 is set from 1 to 100 per packet, including all values and subranges in between, such as 5 cycles, 10 cycles, 20 cycles, 30 cycles, 40 cycles, 50 cycles, 60 cycles, 70 cycles, 80 cycles, 90 cycles, 100 cycles, etc. In some embodiments, the cycle count 420 is 1 to 5 cycles, 1 to 10 cycles, 1 to 25 cycles, 10 to 20 cycles, 1 to 40 cycles, 40 to 50 cycles, 1 to 60 cycles, 50 to 60 cycles, 1 to 80 cycles, 1 to 100 cycles, 50 to 100 cycles, 1 to 1,000 cycles or 1 to 2,000 cycles, including all values and subranges in between.

The packet duration is determined by the cycle count, among other factors. Typically, the higher the cycle count, the longer the packet duration and the larger the quantity of energy delivered. In some embodiments, packet durations are in the range of approximately 50 to 1000 microseconds, such as 50 μs, 60 μs, 70 μs, 80 μs, 90 μs, 100 μs, 125 μs, 150 μs, 175 μs, 200 μs, 250 μs, 100 to 250 μs, 150 to 250 μs, 200 to 250 μs, 500 to 1000 μs to name a few. In other embodiments, the packet durations are in the range of approximately 100 to 1000 microseconds, such as 150 μs, 200 μs, 250 μs, 500 μs, or 1000 μs.

In some embodiments, the number of packets delivered during treatment, or packet count, may include 50 to 280 packets including all values and subranges in between, such as 50, 60, 70, 80, 90, 100, 50-100, 80-100, 100-110, 110, 120, 130, 140, 150, 100-150, 160, 170, 180, 190, 200, 210, 220, 230, 240, 250, 260, 270, and 280 packets.

TABLE 2
Example parameter combinations include:

| | | | Packet | Minimum # | | |
|---|---|---|---|---|---|---|
| Voltage | Frequency | | duration | of Packets | Penetration | |
| 3500 V | 500 | kHz | 250 μs | 200 | 0.1-1 | cm |
| 5000 V | 5 | kHz | 200 μs | 10-20 | 0.5-2 | cm |
| 6000 V | 300 | kHz | 500 μs | 100 | 3-5 | cm |
| 3000 V | 500 | kHz | 250 μs | 25-50 | 0.5-2 | cm |
| 2500 V | 300 | kHz | 150 μs | 100 | 0.5-2 | cm |
| 2500 V | 500 | kHz | 100 μs | 50 | 0.5 | cm |
| 2500 V | 600 | kHz | 100 μs | 20 | 0.05-0.1 | cm |

### E. Rest Period/Inter-Packet Delay

In some embodiments, the time between packets, referred to as the rest period or interpacket delay 406, is set between about 0.1 seconds and about 5 seconds, including all values and subranges in between. In other embodiments, the rest period 406 ranges from about 0.001 seconds to about 10 seconds, including all values and subranges in between, such as 1 second, 2 seconds, 3 seconds, 4 seconds, 5 seconds, 6 seconds, 7 seconds, 8 seconds, 9 seconds, 10 seconds, etc. In some embodiments, the rest period 406 is approximately 3-5 seconds. In particular, in some embodiments the signal is synced with the cardiac rhythm so that each packet is delivered synchronously within a designated period relative to the heartbeats, thus the rest periods coincide with the heartbeats. In other embodiments wherein cardiac synchronization is utilized, the rest period 406 may vary, as the rest period between the packets can be influenced by cardiac synchronization, as will be described in later sections.

### F. Switch Time and Dead Time/Inter-Cycle Delay

A switch time is a delay or period of no energy that is delivered between the positive and negative peaks of a biphasic pulse. In some embodiments, the switch time ranges between about 0 to about 1 microsecond, including all values and subranges in between. In other embodiments, the switch time ranges between 1 and 20 microseconds, including all values and subranges in between. In other embodiments, the switch time ranges between about 2 to about 8 microsecond, including all values and subranges in between.

Delays may also be interjected between each cycle of the biphasic pulses, referred as “dead-time” or inter-cycle delays. Inter-cycle delays occur within a packet, but between cycles or biphasic pulses. This is in contrast to rest periods or inter-packet delays which occur between packets. In other embodiments, the inter-cycle delay 412 is in a range of 0.01 to 0.5 microseconds, 1 to 10 microseconds, 2 to 5 microseconds, 10 to 20 microseconds, 50 to 100 microseconds, 1000 microseconds, 1000 to 1500 microseconds or 1000 microseconds to 100 milliseconds, including all values and subranges in between. In some embodiments, the inter-cycle delay 412 is in the range of 0.2 to 0.3 microseconds. Inter-cycle delays may also be used to define a period between separate, monophasic, pulses within a packet.

Delays of this sort are typically introduced to a packet to reduce the effects of biphasic cancellation within the waveform. Biphasic cancellation is a term used to refer to the reduced induction of cellular modulation in response to biphasic waveforms versus monophasic waveforms, particularly when switch times and dead times are small, such as below 10 μs. In some embodiments, the influence of biphasic cancellation is reduced by introducing switch time delays and dead time. In some instances, the switch time and dead time are both increased together to strengthen the effect. In other instances, only switch time or only dead time are increased to induce this effect.

In some embodiments, the switch time duration is adjusted such that the degree of therapy effect relative to distant cell effects is optimized for the target of the therapy. In some embodiments, the switch time duration or dead time duration is minimized to decrease distant muscle cell contractions, with lesser local therapy effect. In other embodiments, the switch time duration is extended to increase the local therapy effect, with potential additional distant muscle cell contractions. In some embodiments, the switch time or dead time duration are extended to increase the local therapy effect, and the use of neuromuscular paralytics are employed to control the resulting increase in muscle contraction. In some embodiments, switch time duration is 10 ns to 2 μs, while in other embodiments, the switch time duration is 2 μs to 20 μs. In some instances, when cell modulation is targeted in a way where transmembrane potential manipulation is not the primary mechanism needed to evoke the targeted treatment effects, the switch time and dead time delays are minimized to less than 0.1 μs or to 0 μs. This elimination of delays minimizes the peripheral, non-targeted treatment effects such as skeletal muscle contraction or cardiac muscle action potential and contraction.

Another benefit of utilizing switch time and the dead time delays to increase treatment effects for biphasic waveforms is a reduction in generator demands, whereby the introduction of pauses will enable stronger treatment effects without requiring asymmetric/unbalanced pulse waveforms. In this case, unbalanced waveforms are described as those that are monophasic, or have an unbalanced duration or voltage or combination in one polarity relative to the other. In some cases, unbalanced means that the integral of the positive portions of the waveform are not equal to the integral of the negative portions of the waveform. Generators capable of delivering unbalanced waveforms have a separate set of design considerations that are accounted for thereby increasing potential generator complexity.

### G. Waveforms

In some embodiments, the waveform has symmetric pulses, such that the voltage and duration of pulse in one direction (i.e., positive or negative) is equal to the voltage and duration of pulse in the other direction. In some embodiments, the waveform has pulses of unbalanced voltages. An unbalanced waveform may result in a more pronounced treatment effect as the dominant positive or negative amplitude leads to a longer duration of same charge cell membrane charge potential. In some embodiments, imbalance includes pulses having pulse widths of unequal duration. In some embodiments, the biphasic waveform is unbalanced, such that the voltage in one direction is equal to the voltage in the other direction, but the duration of one direction (i.e., positive or negative) is greater than the duration of the other direction, so that the area under the curve of the positive portion of the waveform does not equal the area under the negative portion of the waveform. In some embodiments, an unbalanced waveform is achieved by delivering more than one pulse in one polarity before reversing to an unequal number of pulses in the opposite polarity.

### H. Waveform Shapes

Rather than square waves, pulses are sinusoidal in shape in some embodiments. One benefit of a sinusoidal shape is that it is balanced or symmetrical, whereby each phase is equal in shape. Balancing may assist in reducing undesired muscle stimulation. It may be appreciated that in other embodiments the pulses have decay-shaped waveforms.

It may be appreciated that generating larger lesions (e.g. larger than approximately 1×1×1 cm³) may involve different parameter values to obtain the same or similar lesion characteristics and/or immune response. In some embodiments, a lesion having a size of approximately 1.8×1.8×2.4 cm³ may be achieved by a waveform that is produced from a combination of parameter values that includes the following: voltage=3300V, fundamental frequency=100 kHz, number of biphasic pulses (i.e., cycles) per packet=10 cycles, inter-cycle delay=1000 microseconds, number of packets=100 packets and inter-packet delay=3 seconds. In some embodiments, a lesion having a size of approximately 3×3×4 cm³ may be achieved by a waveform that is produced from a combination of parameter values that includes the following: voltage=6000V, fundamental frequency=100 kHz, number of biphasic pulses (i.e., cycles) per packet=10 cycles, inter-cycle delay=1000 or 2000 microseconds, number of packets=100 packets and inter-packet delay=1, 3, 5 or 10 seconds.

It may be appreciated that in some embodiments, the waveform is generated as a function of electric current rather than voltage. For example, doses generated using 3000V or 3300V are delivered into environments of 150-300 ohm (generating 10-20 A for 3000V and 11-22 A for 3300V). In some embodiments, the electric current is 10 A, 15 A, 20 A, 25 A, 30 A, 35 A, 40 A, 45 A, 50 A, 55 A, 60 A, 65 A, 70 A, etc, and all ranges in between. It may be appreciated that in some instances, such as when using 100 kHz treatments that can be delivered with paralytic, 70 A may be the upper limit.

Energy delivery may be actuated by a variety of mechanisms, such as with the use of an actuator 132 on the instrument 102 or a foot switch operatively connected to the generator 104. Such actuation typically provides a single energy dose. The energy dose is defined by the number of packets delivered and the voltage of the packets. Each energy dose delivered to the target tissue maintains the temperature at or in the target tissue below a threshold for thermal ablation, particularly thermal ablation or denaturing of stromal proteins in the basement membrane or deeper submucosal extracellular protein matrices. In addition, the doses may be titrated or moderated over time so as to further reduce or eliminate thermal build up during the treatment procedure. Instead of inducing thermal damage, defined as protein coagulation at sites of danger to therapy, the energy dose provides energy at a level which induces treatment of the condition, such as cancer, without damaging sensitive tissues.

As used herein, the terms “about” and/or “approximately” when used in conjunction with numerical values and/or ranges generally refer to those numerical values and/or ranges near to a recited numerical value and/or range. In some instances, the terms “about” and “approximately” can mean within ±10% of the recited value. For example, in some instances, “about 100 [units]” can mean within ±10% of 100 (e.g., from 90 to 110). The terms “about” and “approximately” can be used interchangeably.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treatment planning for a patient, the method comprising:
delivering pulsed electric field energy to the patient;
generating an evaluation of antibody production by the patient in response to the delivering of pulsed electric field energy; and
utilizing the evaluation to plan future treatment of the patient.

2. The method of claim 1, wherein the evaluation comprises a quantification of the antibody production.

3. The method of claim 1, wherein the evaluation comprises identification of one or more types of antibodies in the antibody production.

4. The method of claim 1, wherein the pulsed electric field energy comprises packets of biphasic pulses.

5. The method of claim 1, wherein generating an evaluation of antibody production comprises:
obtaining a biospecimen from the patient after providing the treatment to the patient, wherein the biospecimen has antibodies;
combining at least a portion of the biospecimen with at least one target antigen; and
generating a colorimetric solution having an optical density that indicates an amount of antibodies present in the at least a portion of the biospecimen that have bound to the at least one target antigen thereby indicating antibody production.

6. The method of claim 5, wherein generating a colorimetric solution comprises combining tagging antibodies with the amount of antibodies.

7. The method of claim 6, wherein the tagging antibodies are specific to an antibody type IgA, IgM, IgG, or IgE.

8. The method of claim 6, wherein generating the colorimetric solution comprises conjugating the tagging antibodies with an identifying material.

9. The method of claim 8, wherein the identifying material comprises an enzyme.

10. The method of claim 8, wherein generating the colorimetric solution comprises combining a color revealing solution with the conjugated tagging antibodies so as to induce a colorimetric chemical reaction that colors or changes color of the color revealing solution.

11. The method of claim 10, wherein a degree of optical density directly correlates to quantity of tagging antibody.

12. The method of claim 10, further comprising measuring the optical density.

13. The method of claim 5, wherein a higher optical density correlates to a higher amount of antibodies present.

14. The method of claim 5, wherein the at least one target antigen is obtained from the patient.

15. The method of claim 14, wherein the at least one target antigen is obtained from a tumor and the at least one target antigen is a tumor antigen.

16. The method of claim 5, wherein the at least one target antigen is synthetically produced.

17. The method of claim 5, wherein the biospecimen comprises blood.

18. The method of claim 1, wherein the future treatment comprises additional delivery of pulsed electric field energy.

19. A method of determining an immunological status of a patient, the method comprising:
obtaining a biospecimen having antibodies from the patient;
combining at least a portion of the biospecimen with at least one target antigen;
generating a colorimetric solution having an optical density that indicates an amount of the antibodies present in the at least a portion of the biospecimen that have bound to the at least one target antigen; and
determining the immunological status of the patient based at least in part on the amount.

20. The method of clause 19, wherein generating a colorimetric solution comprises combining tagging antibodies with the amount of antibodies, conjugating the tagging antibodies with an identifying material, and combining a color revealing solution with the conjugated tagging antibodies so as to induce a colorimetric chemical reaction that colors or changes color of the color revealing solution.
